(19) United States
(12) Patent Application Publication
(10) Pub. No.: US 2025/0064363 A1
(43) Pub. Date: Feb. 27, 2025

(54) COLLECTION PAD FOR THE SCREENING AND DETECTION OF BIOMARKERS IN MENSTRUAL BLOOD

(71) Applicant: Lilypad Health Inc., Atlanta, GA (US)

(72) Inventors: Rhea Prem, Lynnfield, MA (US); Netra Gandhi, Grovetown, GA (US); Ethan Nathaniel Damiani, Atlanta, GA (US); Girish Hari, Tampa, FL (US)

(21) Appl. No.: 18/809,667

(22) Filed: Aug. 20, 2024

Related U.S. Application Data
(60) Provisional application No. 63/578,048, filed on Aug. 22, 2023.

(51) Int. Cl. A61B 5/15

(52) U.S. Cl. CPC A61B 5/150045; A61B 5/150358; A61B 5/150778; A61B 5/150793

(57) ABSTRACT

A multilayer collection pad includes a fluid permeable top layer configured to be in proximity to a flow of menstrual effluent of a user, the menstrual effluent of a user comprising menstrual blood. The multilayer collection pad also includes a cover layer; and an adhesive layer disposed between the fluid permeable top layer and the cover layer, such that a perimeter of each of the layers is substantially aligned. A collection strip is included that is disposed between the fluid permeable top layer and the adhesive layer. The collection strip includes a tab extending from an edge of the collection strip past the perimeter of each of the layers.

### CROSS-REFERENCE TO RELATED APPLICATIONS

This application claims the benefit of and priority to U.S. Provisional App. No. 63/578,048, filed on Aug. 22, 2023. The entire contents of which are hereby incorporated by reference.

### FIELD

The present disclosure relates to systems, devices, and methods for collecting menstrual blood for screening and detection of biomarkers.

### BACKGROUND

Approximately 1 in 15 women in the United States do not have access to a gynecologist, with minority women being affected disproportionately. Many of these women lack accessibility to procedures such as pap smears and routine blood tests that can help diagnose life threatening diseases. Lack of access to gynecological screening procedures increases the risk of developing serious conditions (e.g., Human Papillomavirus (HPV), cervical cancer, chlamydia, gonorrhea, and human immunodeficiency virus (HIV)) that can be detected and treated earlier. Moreover, available procedures can be invasive and uncomfortable for patients which can often lead to patients avoiding screening procedures.

Menstrual blood contains key biomarkers that can be used to diagnose these ailments early. However, menstrual blood is severely underutilized as a screening tool.

The present disclosure is directed to systems, devices, and methods that collect menstrual blood for extracting health information.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a multilayer collection pad comprising a fluid permeable top layer configured to be in proximity to a flow of menstrual effluent of a user, a cover layer, an adhesive layer, and a collection strip. The menstrual effluent of a user comprising menstrual blood. The adhesive layer is disposed between the fluid permeable top layer and the cover layer, such that a perimeter of each of the layers is substantially aligned. The collection strip is disposed between the fluid permeable top layer and the adhesive layer. The collection strip includes a tab extending from an edge of the collection strip past the perimeter of each of the layers.

In some implementations, the multilayer collection pad further comprises a non-organic layer disposed between the collection strip and the adhesive layer.

In some implementations, the collection strip comprises organic material.

In some implementations, the fluid permeable top layer further comprising a tab extending past the perimeter of each of the plurality of layers.

In some implementations, the adhesive layer comprises a base layer and an adhesive disposed on at least a portion of the base layer. In some implementations, the adhesive comprises hydrophobic material.

In some implementations, the adhesive layer comprises hydrophilic material.

In some implementations, the fluid permeable top layer comprises a number of pores each of the number of pores having a diameter in the range of of 0.1 to 25 micrometer.

In some implementation, the adhesive layer comprises a number of pores each of the number of pores having a diameter in the range of 0.1 to 25 micrometer.

In some implementations, the cover layer covers the adhesive.

In some implementations, the fluid permeable top layer further comprising an indicator, the indicator sized and shaped to demonstrate to the user of the multilayer collection pad an area to fill with menstrual blood.

The disclosed subject matter also includes a kit comprising a multilayer collection pad and a primary container for receiving the collection strip. The multilayer collection pad comprising a fluid permeable top layer configured to be in proximity to a flow of menstrual effluent of a user, a cover layer, an adhesive layer, and a collection strip. The menstrual effluent of a user comprising menstrual blood. The adhesive layer is disposed between the fluid permeable top layer and the cover layer, such that a perimeter of each of the layers is substantially aligned. The collection strip is disposed between the fluid permeable top layer and the adhesive layer. The collection strip includes a tab extending from an edge of the collection strip past the perimeter of each of the layers.

In some implementations, the primary container further comprises a silica desiccant.

In some implementations, the primary container further comprises a plurality of slots to secure at least one collection strip.

In some implementations, the kit further comprises a unique identification code for reviewing a status of the collection pad.

In some implementations, the collection strip further comprises a unique identification code.

In some implementations, the unique identification code comprising a serial code.

In some implementations, the unique identification code comprising a QR code.

In some implementations, the kit further comprises readable and graphical instructions including application of the collection pad; usage of the collection pad, the instructions for usage of the collection pad including a graphic illustrating an area of the collection pad to stain with menstrual blood of a user for laboratory testing; and shipping of the collection strip.

The disclosed subject matter also includes a method of using a multilayer collection pad comprising applying a multilayer collection pad; staining the collection pad; separating the collection strip from the layers of the collection pad; and receiving results of an analysis of the collection strip.

In some embodiments, the method further comprises determining where the collection pad is sufficiently stained for testing.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementation. In the drawings, like reference characters generally refer to like features, functionally and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawing in which:

FIG. 1 is a view of one implementation of a multilayer collection pad.

FIG. 2 is an exploded assembly view of the implementation of the multilayer collection pad of FIG. 1.

FIGS. 3A-B are views of the implementation of a top layer of the multilayer collection pad of FIGS. 1 and 2

FIGS. 4A-B are views of one implementation of a collection strip.

FIGS. 5A-B are views of a second implementation of a collection strip.

FIG. 6 is a view of a second implementation of a multilayer collection pad.

FIG. 7 is a side view of the implementation of the multilayer collection pad of FIG. 6.

FIG. 8 illustrates an implementation of a kit including a multilayer collection pad.

FIG. 9 illustrates an implementation of user instructions for using a multilayer collection pad.

FIG. 10 is a method of using an implementation of a multilayer collection pad.

FIGS. 11A-D are various views of a display screen of an implementation of an application.

FIGS. 12A-B are views of an implementation of a housing for lateral flow assay testing.

FIG. 12C is a perspective view of the housing of FIGS. 12A-B with a collection strip.

FIG. 13A is an isometric top down view of a partially unassembled third implementation of a multilayer collection pad.

FIG. 13B is an isometric bottom up view of the partially unassembled implementation of the multilayer collection pad of FIG. 13A.

FIG. 13C is a top view of an assembled implementation of the multilayer collection pad of FIGS. 13A-B.

FIG. 13D is a bottom view of the assembled implementation of the multilayer collection pad of FIG. 13C.

FIGS. 14A-B are various views of an implementation of an adhesive layer of a multilayer collection pad.

FIGS. 15A-B are various views of an implementation of adhesive on an adhesive layer of a multilayer collection pad.

FIGS. 16A-C are various views of an implementation of a primary container for holding a collection strip of a multilayer collection pad.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

References herein to positions of elements (e.g., “top”, “bottom”) are merely used to describe the orientation of various elements in the FIGURES. It should be noted that the orientation of various elements may differ according to other exemplary implementations, and that such variations are intended to be encompassed by the present disclosure.

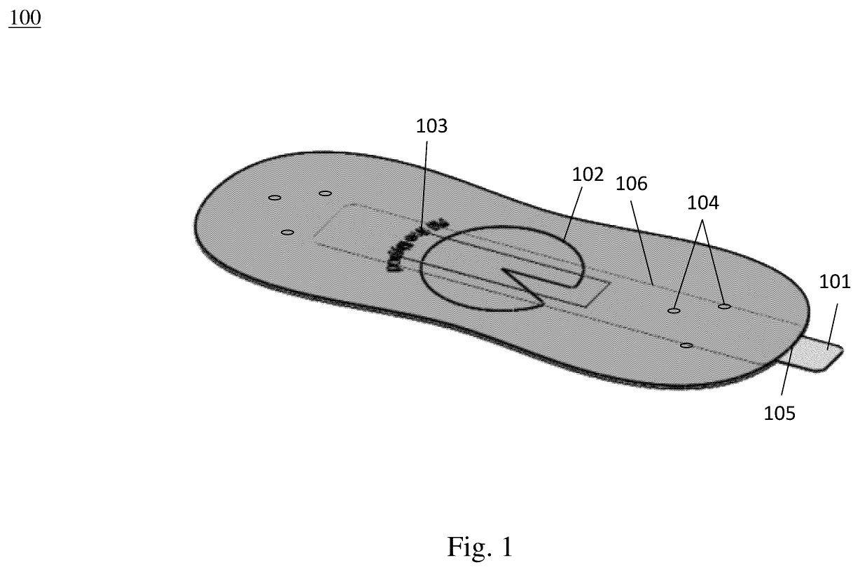

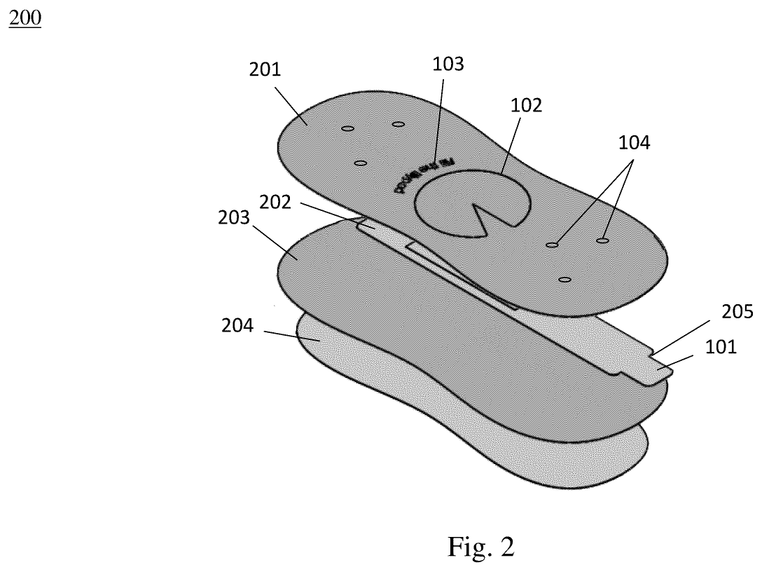

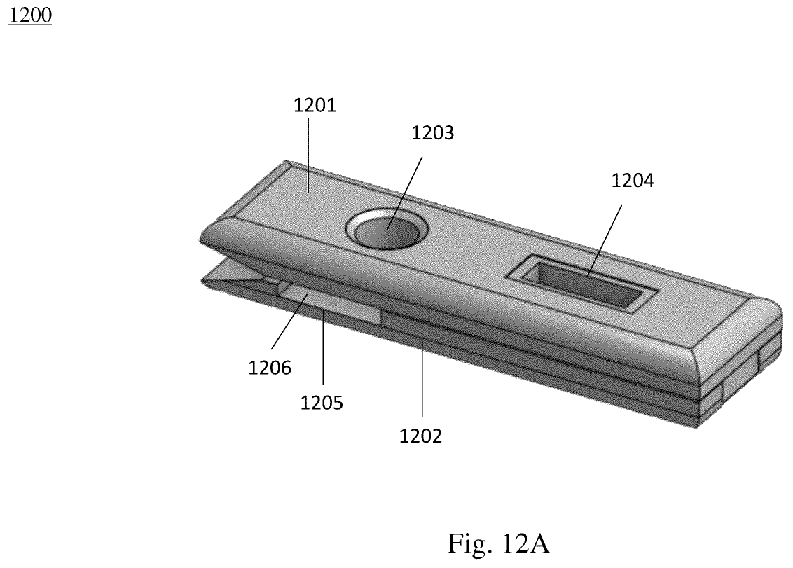

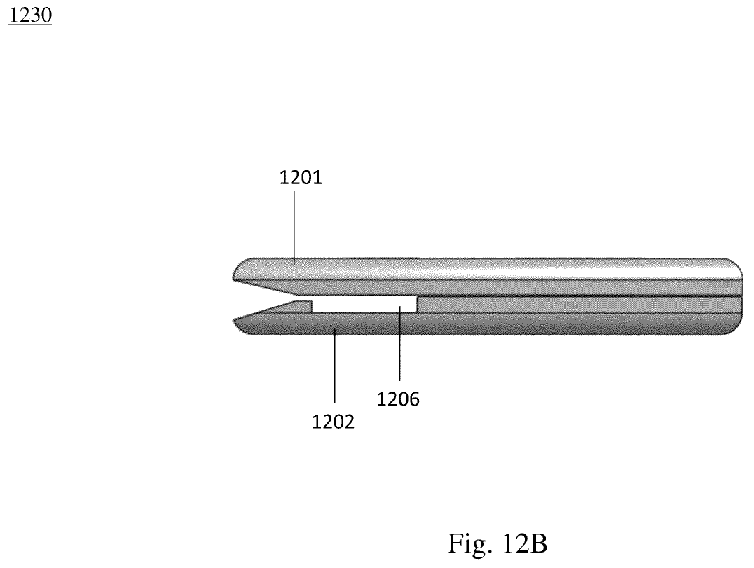

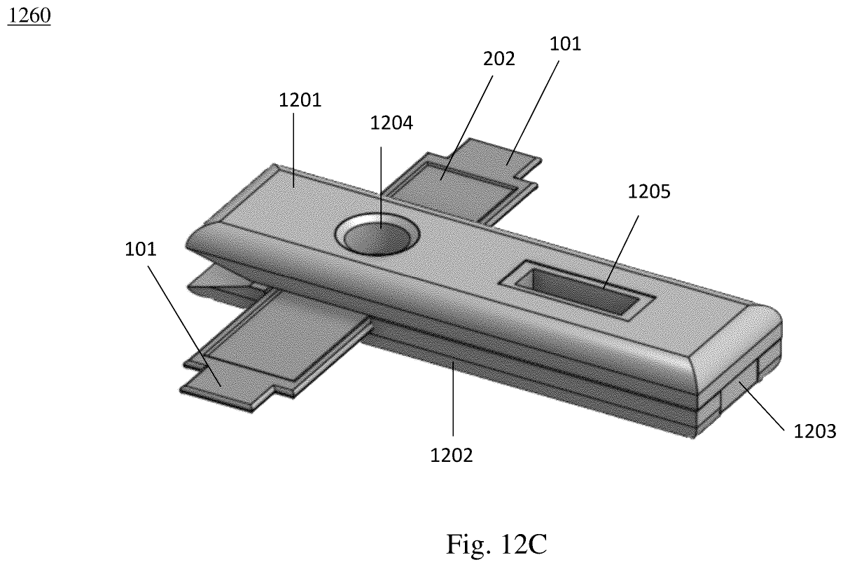

Referring now to FIG. 1, and in brief overview, and with additional reference to FIG. 2, a view and an exploded assembly view of an implementation of a multilayer collection pad are shown that includes a top layer 201, a collection strip 202, an adhesive layer 203, and a cover layer 204. The top layer 201 can be configured to be in proximity to a flow of menstrual effluent of a user and be fluid permeable to menstrual blood. The adhesive layer 203 can be disposed between the top layer 201 and the cover layer 204. The collection strip 202 can be disposed between the top layer 201 and the adhesive layer 203. The collection strip 202 can include a tab 101 extending from at least a portion of an edge 205 of the collection strip 202. The tab 101 can extend past the perimeter of each of the layers. In some implementations, the tab 101 of the collection strip 202 is a plurality of tabs which can extend from opposite ends of the collection strip 202 (illustrated in FIG. 12C). The collection strip 202 and the cover layer 204 can be configured to be removable from the multilayer collection pad.

The top layer 201, the adhesive layer 203, and the cover layer 204 can form a multilayer stack with the collection strip 202 such that the perimeters of at least the layers 201, 203, 204 are substantially aligned. The top layer 201 and the adhesive layer 203 can be coupled to each other. In some implementations, the top layer 201 and the adhesive layer 203 can be coupled to each other (e.g., via heat sealing) along a portion of their perimeter such that an unsealed portion 105 provides an opening through which the tab 101 of the collection strip 202 extends through.

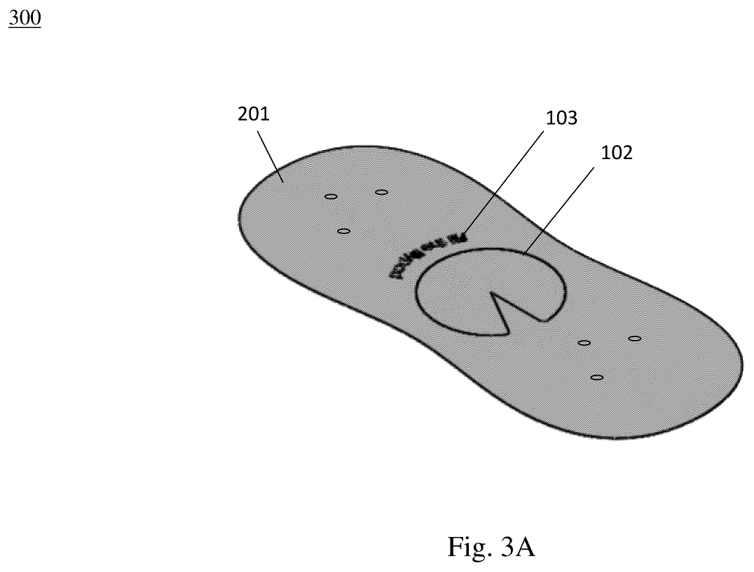

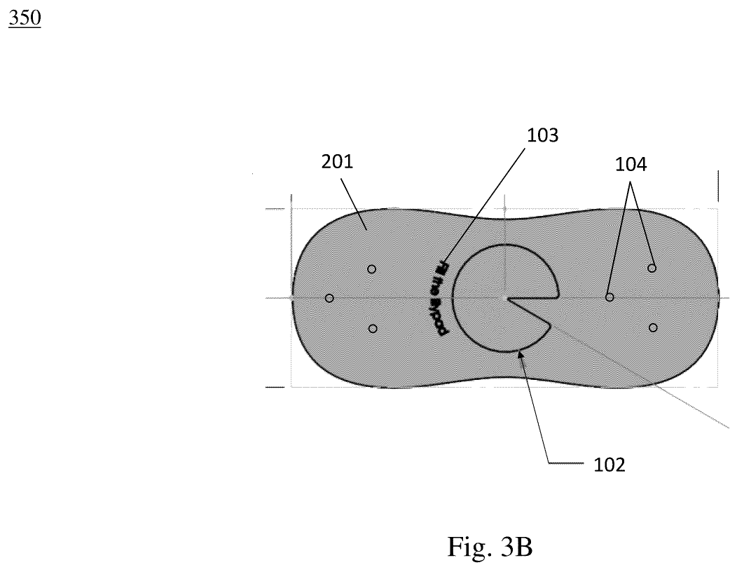

Still referring to FIG. 1, and in greater detail, and with additional reference to FIGS. 2, and 3A-B, the top layer 201 can be a fluid permeable top layer that makes contact with the user's skin and receives the flow of menstrual effluent of the user. The top layer 201 can filter menstrual effluent (e.g., menstrual blood, vaginal secretions) to ensure that menstrual blood stains (i.e., moistens, saturates) the collection strip 202. Menstrual blood can saturate the collection strip 202 and the surrounding layers. Menstrual blood can include whole blood, plasma, DNA, RNA, proteins, antigens, antibodies, cells, and other disease biomarkers.

The top layer 201 can be formed of a material that allows fluid communication between the menstrual blood of the user and the collection strip 202 disposed between the top layer 201 and the adhesive 203 layer. The top layer 201 can comprise a material that is porous and defines a number of pores through which menstrual blood can flow through so that blood can contact and stain the collection strip 202. Excess menstrual blood that flows through the top layer 201 (i.e., blood that does not stain the collection strip 202) can flow through to the adhesive layer 203.

The top layer 201 can comprise a number of pores 104 with pore sizes selected to allow menstrual blood passage through the top layer while preventing the passage of eggs, mucus, blood clots, other non-blood related components, and other non-fluid effluent. In some implementations, the top layer 201 is a non-woven polypropylene layer. The number of pores 104 can refer to the spaces or gaps between the fibers of the non-woven layers. In some implementations, the number of pores 104 can be different shapes such as a circle, rectangle, rectangle with rounded corners, triangle, or an irregular shape. In some implementations, the number of pores 104 are arranged in a patterned manner (e.g., grid-like fashion). In other implementations, the number of pores 104 are arranged in a non-patterned manner (e.g., randomly distributed or irregularly distributed). In some implementations, each of the number of pores 104 are the same diameter. In some implementations, each of the number of pores 104 has a diameter in the range of 0.1 to 25 micrometer. In other implementations, each of the number of pores 104 has a diameter in the range of 12.5 to 25 micrometers. In still other implementations, each of the number of pores 104 has a diameter in the range of 25 to 75 micrometers. In further implementations, each of the number of pores 104 has a diameter in the range of 50 to 100 micrometers. In still further implementations, each of the number of pores 104 has a diameter in the range of 0.1 to 100 micrometers. In some implementations, the lower range of diameters of the number of pores 104 can be chosen to prohibit the passage of microclots (approximately between 1 to 60 microns) through the top layer 201.

In some implementations, the top layer 201 is made of one or more materials (e.g., a combination thereof), including but not limited to polyester, cotton, polyethylene, polypropylene, a blend of both polypropylene and polyethylene, bamboo, rayon, viscose rayon, wood pulp, polyethylene terephthalate, hot melt adhesive, sodium polyacrylate, ethylene-vinyl acetate, PEG-7 glyceryl cocoate, polyoxyalkylene substituted chromophore, titanium dioxide, PEG-10 cocoate, PEG sorbitol hexaoleate, PEG hydrogenated castor oil trilaurate, color pigments, plant based PLA, hydrocarbon resin, styrene/isoprene copolymer, synthetic beeswax, kaolin, PEG-10 oleate, sorbitan oleate, diethylhexyl sodium sulfosuccinate, and ethylene carbonate.

The top layer 201 can further include an indicator 102. The indicator 102 can include a marked outlined perimeter that is sized and shaped to demonstrate to the user an area to fill with menstrual blood. The size of the area enclosed by the outlined perimeter can correspond to the degree of saturation of the top layer 201 pad necessary to stain the collection strip 202 with sufficient menstrual blood for processing and biomarker testing. In some implementations, the indicator 102 is ovular in shape. In other implementations, the indicator 102 is rectangular in shape. In still other implementations, the indicator 102 is an irregular shape. The indicator 102 can provide a visible indicator to the user which allows the user to determine if the collection strip 202 is stained sufficiently for processing and biomarker testing. In this way, the user does not have to remove the collection strip 202 from the multilayer collection pad or separate the layers of the multilayer collection pad to view the collection strip 202. Furthermore, displaying a visible indicator 102 on the top layer 201 can reduce the chance of damaging the collection strip 202. The top layer 201 can further include a printed design 103. In some implementations, the printed design 103 is an instruction to the user. In some implementations, the printed design 103 is a logo.

The top layer 201 can include padding or cushioning to provide additional comfort to the user. The top layer 201 can further include multiple layers of porous material to provide a gradient of filtration.

The multilayer collection pad can further comprise a collection strip boundary 106. The collection strip boundary 106 can include a three-sided outline that extends through the layers above and below the collection strip 202 (e.g., top layer 201, adhesive layer 203). The collection strip boundary 106 can be sized and shaped to surround three edges of the collection strip 202 when it is placed between the layers of the collection pad (e.g., top layer 201 and the adhesive layer 203). By extending through all the layers of the multilayer collection pad, the collection strip boundary 106 can define a pocket configured to hold the collection strip 202 in place, ensuring proper alignment and preventing shifting of the collection strip 202. Two sides of the three-sided outline can be substantially parallel to each other, and the distance between them can correspond to the width of the collection strip 202, with an additional tolerance. These sides can extend from the unsealed portion 105 to a perpendicular edge of the collection strip 202, with an additional tolerance. The third side of the three-side outline can be perpendicular to the other two sides and connects them, completing the outline. The tolerance ensures that the collection strip is not tightly secured between the layers, allowing it to be slid into and out of the multilayer collection pad. It should be understood that depending on the shape and dimensions of the collection strip 202, the collection strip boundary 106 may be a different outline to correspond to the geometry of the collection strip 202.

Still referring to FIG. 1, and in greater detail, and with additional reference to FIG. 2, the adhesive layer 203 can include a base layer and an adhesive. The adhesive can be disposed on at least a portion of the base layer and can be disposed so that it is proximal to a fabric of the user (e.g., undergarment, menstrual pad beneath the multilayer collection pad) when the user applies the multilayer collection pad. The adhesive layer 203 can adhere the multilayer collection pad to the menstrual pad or undergarment of a user. In some implementations the adhesive layer 203 at least partially absorbs the menstrual blood of the user.

In some implementations, the adhesive layer 203 is a fluid impermeable layer that prevents excess menstrual blood from flowing through the adhesive layer. In other implementations, the adhesive layer 203 can be formed of a material that allows fluid communication between the adhesive layer 203 and the menstrual pad of the user. In those implementations, the adhesive layer 203 is a fluid permeable layer that allows excess menstrual blood (i.e., blood that does not stain the collection strip 202) to flow through the layer. In this way, clotting, user discomfort, and oversaturation of the multilayer collection pad are prevented by allowing excess menstrual blood to flow through the adhesive layer 203. In some implementations, the adhesive layer 203 contains fluid permeable regions and fluid impermeable regions.

The adhesive layer 203 (e.g., the base layer of the adhesive layer 203) can be formed of a material that is porous and defines a number of pores through which menstrual blood can flow through. Excess menstrual blood that flows through the adhesive layer 203 can contact and be absorbed by a menstrual pad of the user disposed below the adhesive layer 203. In some implementations, the adhesive layer 203 comprises a number of pores 104 with pore sizes selected to allow menstrual blood passage through the adhesive layer 203. In some implementations, the adhesive layer 203 is a non-woven polypropylene layer. In those implementations, the number of pores 104 refer to the spaces or gaps between the fibers of the non-woven polypropylene layer. In other implementations, the number of pores 104 can be different shapes such as a circle, rectangle, rectangle with rounded corners, triangle, or an irregular shape. In some implementations, the number of pores 104 are arranged in a patterned manner (e.g., grid-like fashion). In other implementations, the number of pores 104 are arranged in a non-patterned manner (e.g., randomly distributed or irregularly distributed). In some implementations, each of the number of pores 104 are the same diameter. In some implementations, each of the number of pores 104 has a diameter in the range of 0.1 to 25 micrometer. In other implementations, each of the number of pores 104 has a diameter in the range of 12.5 to 25 micrometers. In still other implementations, each of the number of pores 104 has a diameter in the range of 25 to 75 micrometers. In further implementations, each of the number of pores 104 has a diameter in the range of 50 to 100 micrometers. In still further implementations, each of the number of pores 104 has a diameter in the range of 0.1 to 100 micrometers.

In some implementations, the adhesive layer 203 can be made of one or more materials (e.g., a combination thereof), including but not limited to polyester, cotton, polyethylene, polypropylene, a blend of both polypropylene and polyethylene, rayon, bamboo, viscose rayon, wood pulp, polyethylene terephthalate, hot melt adhesive, sodium polyacrylate, ethylene-vinyl acetate, PEG-7 glyceryl cocoate, polyoxyalkylene substituted chromophore, titanium dioxide, PEG-10 cocoate, PEG sorbitol hexaoleate, PEG hydrogenated castor oil trilaurate, color pigments, plant based PLA, hydrocarbon resin, styrene/isoprene copolymer, synthetic beeswax, kaolin, PEG-10 oleate, sorbitan oleate, diethylhexyl sodium sulfosuccinate, and ethylene carbonate.

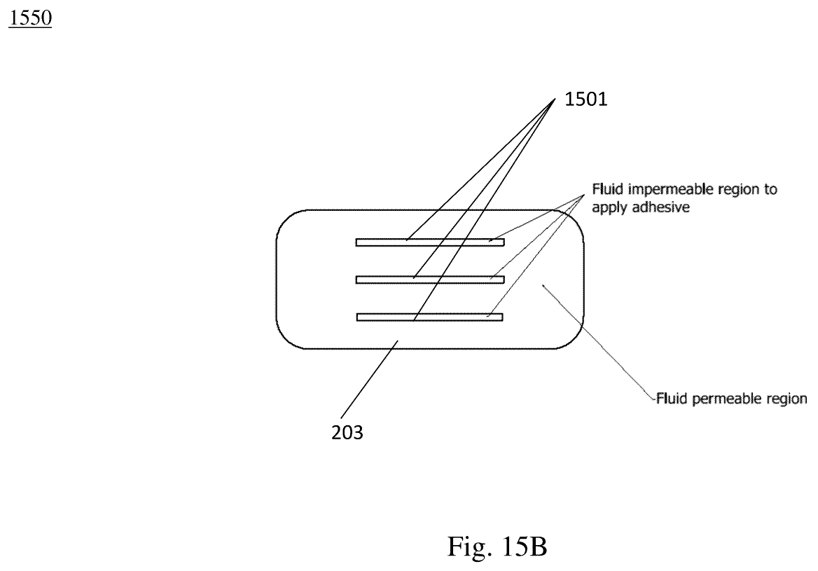

In some implementations, the base layer of the adhesive layer 203 is made of polyethylene. In other implementations, the base layer of the adhesive layer 203 is made of polypropylene. In some implementations, the base layer of the adhesive layer 203 comprises a hydrophilic material. The hydrophilic material can enable blood to flow through the base layer to the menstrual pad of the user which can mitigate clotting, user discomfort, and oversaturation of the multilayer collection pad. In some implementations, the base layer of the adhesive layer 203 comprises a fluid permeable material which can enable blood to flow through the base layer to the menstrual pad of the user. In some implementations, the adhesive of the adhesive layer 203 is a synthetic resin adhesive. In some implementations, the adhesive comprises a hydrophobic material. In those implementations, the hydrophobicity of the adhesive can prevent shifting of the multilayer collection pad during use. In addition, the hydrophobicity of the adhesive can direct excess menstrual blood to flow around the adhesive. In this way, the integrity of the adhesive, including the stickiness or adhesiveness, can be maintained during use of the multilayer collection pad. In some implementations, the adhesive is one or more adhesive strips 1501 disposed on the base layer (as illustrated in FIGS. 15A-B). In some implementations, the shape of the adhesive is circular, rectangular, or irregularly shaped.

The top layer 201 and/or the adhesive layer 203 can be configured to create an even or distributed staining on the collection strip 202 to optimize and maximize viable test samples. By way of example, the top layer 201 and/or the adhesive layer 203 can be configured as a surge layer to distribute fluid across the collection strip 202. A surge layer can be made from highly absorbent materials, such as superabsorbent polymers (SAP) or specialized nonwoven fabrics.

Still referring to FIG. 1, and in greater detail, and with additional reference to FIG. 2, the cover layer 204 can be a removable and disposable layer of the multilayer collection pad. The cover layer 204 can serve as a protective layer to protect the adhesive on the adhesive layer 203 from wear that can affect the adhesive properties of the adhesive layer. The cover layer 204 can cover the adhesive of the adhesive layer 203 up until use and can be peeled off to expose the adhesive for application of the multilayer collection pad. In some implementations, the cover layer 204, is the same size and shape as the adhesive layer 203. In other implementations, the cover layer 204 is a smaller size than the adhesive layer 203. In those implementations, the cover layer 204 is sized and shaped to cover the adhesive of the adhesive layer 203.

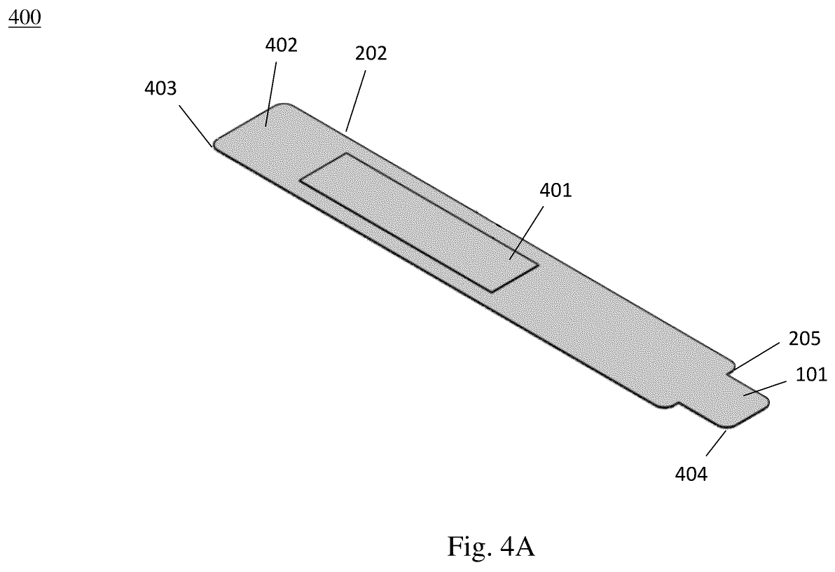

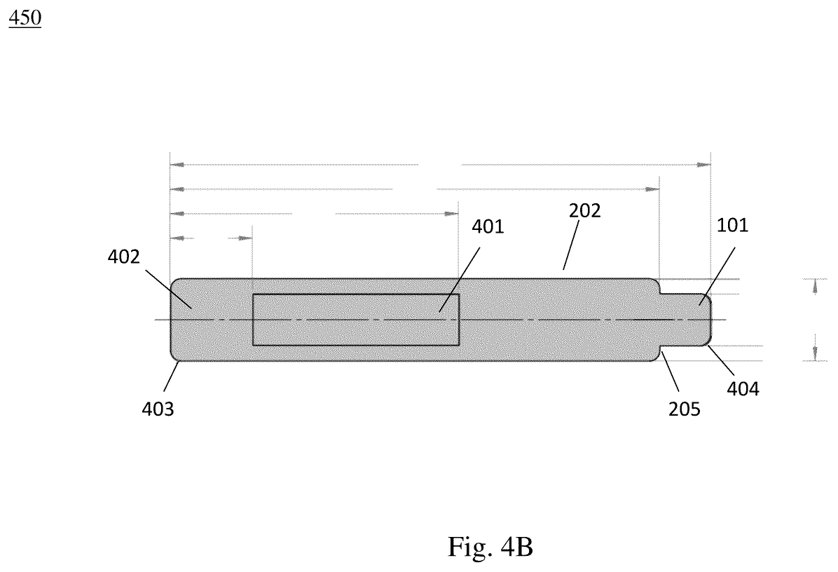

Referring now to FIGS. 4A-B, and with additional reference to FIGS. 1 and 2, views of an implementation of a collection strip 202 are shown that includes a body 402 of the collection strip 202 and a sample membrane 401 disposed on the body 402 of the collection strip 202. The collection strip 202 can collect menstrual blood, DNA, antibodies, cells, and other disease biomarkers. The sample membrane 401 can be shaped and sized so that it covers at least a portion of the area of the body 402 of the collection strip 202. In some implementations, the body 402 of the collection strip 202 can be rectangular in shape with corners 403 that are curved. In other implementations, the body 402 of the collection strip 202 can be different shapes such as rectangular, circular, triangular, or irregularly shaped. The sample holder area 401 can be formed of a material that undergoes staining when fluid contacts it. This property enables the collection strip 202 to preserve the dried blood sample and maintain its viability for up to 180 days. This grants the user with the flexibility to deliver their collection strip 202 for processing and testing. Due to the variability of menstrual cycles, this window of time can be beneficial for menstruating populations as it takes into account the busy schedules in the user's and gives them ample time for their sample to arrive to a laboratory for processing and testing.

The collection strip 202 can be used to test one or more biomarkers including, but not limited to, antibodies, antigens, enzymes, hormones, DNA, RNA, Viruses, bacteria, proteins, and other molecules. The collection strip 202 can test/measure one or biomarkers including, but not limited to, DNA viruses, RNA viruses, C-Reactive Protein, hormones (e.g., estrogen, progesterone, follicle-stimulating hormone (FSH), luteinizing hormone (LH)), antibodies, antigens, NK cells, hemoglobin A1C (HbA1c), lipids, human DNA, human RNA, HPV (High-Risk genotypes 16, 18, 31, 33, 35, 39, 45, 51, 52, 56, 58, 59, 66, 68), HIV 1&2, Dengue, Zika, Herpes, Chlamydia, Gonorrhea, LDL, HDL, TSH, Free T3, Free T4, T3, T4, Anti-TPO, Anti-TG, T Uptake, TSH3-Ultra, AFP, AMH, DHEAS, Estradiol, Free BhCG, FSH, hCG, LH, Progesterone, Prolactin, Testosterone, CA 125, CA 15-3, CA 19-9, Calcitonin, CEA, Complexed PSA, PSA, Serum HER-2/neu, BR 27.29, Homocysteine, HCV, syphilis, trichomoniasis, CMV IgG, CMV IgM, Herpes-1 IgG, Herpes-2 IgG, Rubella IgG, Rubella IgM, Toxoplasma IgG, Toxoplasma IgM, C-Peptide, Insulin, ELF, Active B12, Ferritin, Folate, RBC Folate, Vitamin B12, Anti-CCP, Intact PTH, BNP proteins, CK-MB, CK, NT-proBNP, Troponin proteins, Myoglobin, TnI-Ultra, Anti-HBS2, HAV IgM, HAV Total, HBc IgM, HBc Total, HBe Ag, HBe Ag Confirmatory, HBs AgII, Beta Crosslaps, Osteocalcin, Cyfra 21-1, HE4, ACTH, HCG+B, PIGF SFIt-1, SHBG, Anti-TSHR, Free Androgen Index, Thyroglobulin, Digoxin, hGH, IGF-1, IGF BP3, HSV IgGs, IL-6, Procalcitonin, Cyclosporine, Tacrolimus, Ferritin, Iron, sTfR, Transferrin, UIBC, Total Cholesterol, Triglycerides, Fructosamine, Glucose, HbA1c, Microalbumin, Cortisol, Free Testosterone, Thyroid Peroxidase Antibodies, tTG-IgA, DGP-IgG, total IgA, Triglycerides, HbA1c, Vitamin D, hs-CRP, IgE, IgG, IgM, 25-OH D (Vitamin D3), p24, albumin, ALP, ALT AST, bilirubin, chloride, calcium, alkaline phosphatase, BUN, sodium, potassium, CO2, eGFR, an Complete Blood Count. A single collection strip 202 can be used to test for one or more biomarkers.

The collection strip 202 can be analyzed with one or more testing methods including, but not limited to, High-Performance Liquid Chromatography (HPLC), Polymerase Chain Reaction (PCR), qPCR, rapid/antibody testing, immunoassays/immunoassay systems, and Enzyme-Linked Immunosorbent Assay (ELISA). The collection strip 202 can undergo other testing such as genetic DNA/RNA testing, antigen/antibody testing, and testing for volatile compounds. In some implementations, the blood collected on the collection strip can be tested immediately (i.e., while the blood is in its hydrated form). In some implementations, the blood collected on the collection strip 202 can be allowed to dry before testing to create a dried blood stain. The dried blood stain on the collection strip 202 can be used to test for diseases including, but not limited to, sexually transmitted diseases (STDs), sexually transmitted infections (STIs), genetic disorders, cancers, viruses, bacterial infections, hormone imbalances, metabolic diseases, deficiency diseases, and other physiological diseases. The collection strip 202 can be used to test for and assess various other health related metrics.

To prepare a collection strip 202 for biomarker testing, a sterile hole punch tool can be used to punch out smaller samples of the sample membrane 401. The punched out samples can be deposited into an eluent solution to extract target biomarkers into the solution for processing. In following, one or more testing methods can be applied to the solution for biomarker testing. The bottle or container containing the solution can be labeled with the corresponding unique identification code to link the test results back to the user. The test results of the sample can be recorded and transmitted back to the user via a digital platform (e.g., website, email, mobile device application) or other means.

In some implementations, the sample membrane 401 is Whatman 903 filter paper which is manufactured from 100% pure cotton linters. In some implementations, the body 402 of the collection strip 202 is made of polyester. In other implementations, the body 402 of the collection strip 202 is made of plastic. In some implementations, the collection strip 202 comprises an organic material. In some implementations, the collection strip 202 comprises an inorganic material. In some implementations, collection strip 202 comprises one or more materials (e.g., a combination thereof), including but not limited to bamboo, polyethylene, polypropylene, polylactic acid, Polyhydroxyalkanoates, bio-based PET, Algal bioplastics, cellulose-based plastics, and starch-based plastics.

In some implementations, one or more additional layers can be disposed between at least one of the following: 1) the collection strip 202 and the adhesive layer 203, 2) the collection strip 202 and the top layer 201. In some implementations, the additional layer can be made of a non-organic material. In some implementations, the additional layer can be a surge layer which can be made from highly absorbent materials, such as superabsorbent polymers (SAP) or specialized nonwoven fabrics. The additional layer can prevent the binding between the collection strip 202 and the other layers during saturation of the multilayer collection pad. This can also facilitate an easier extraction of the collection strip 202 once it is sufficiently stained. The additional layers can help prevent tearing of the collection strip 202 when the user separates the collection strip 202 from the stained layers of the multilayer collection pad. In some implementations, the one or more additional layers disposed between the collection strip 202 and the adhesive layer 203 can be made of absorbent materials. In some implementations, the one or more additional layers disposed between the collection strip 202 and the adhesive layer 203 can be layered so that menstrual blood is absorbed and flow to through the adhesive layer 203, while preventing the flow of menstrual blood from a fabric (e.g., menstrual pad) disposed below the multilayer collection pad to flow back through the adhesive layer 203 and towards the collection strip 202.

The tab 101, extending from at least a portion of an edge 205 of the collection strip 202, can be configured to be graspable by a user. This can allow the user to extract the collection strip 202 from the multilayer collection pad by pulling on the tab 101. The tab 101 can be rectangular in shape with corners 404 that are curved.

In some implementations, the multilayer collection pad is smaller than commercially available menstrual pads so that it only partial covers the menstrual pad when the multilayer collection pad is applied. In those implementations, the multilayer collection pad can be applied centrally on a menstrual pad to absorb as much of the menstrual blood as possible. In some implementations, the multilayer collection pad is the same size as commercially available menstrual pads. In some implementations, the multilayer collection can be applied directly onto a fabric worn by the user (e.g., undergarment).

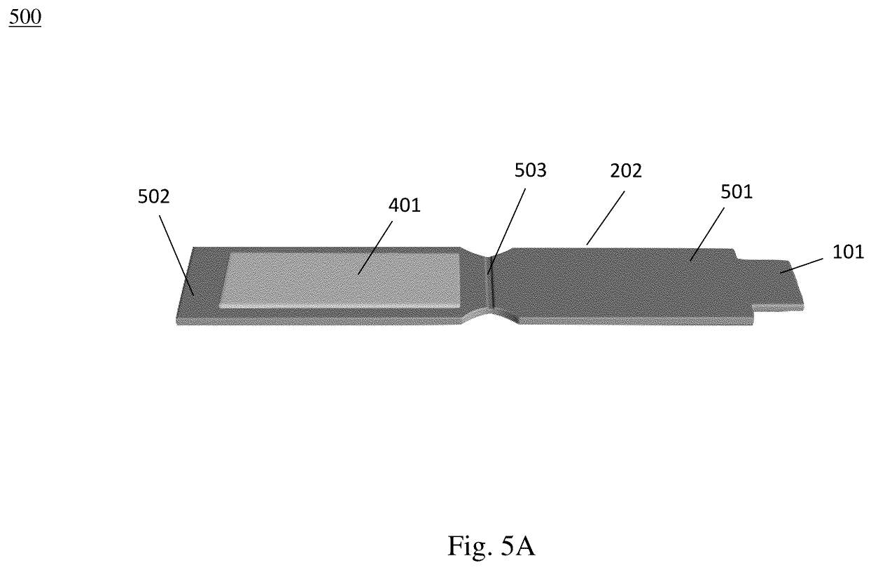

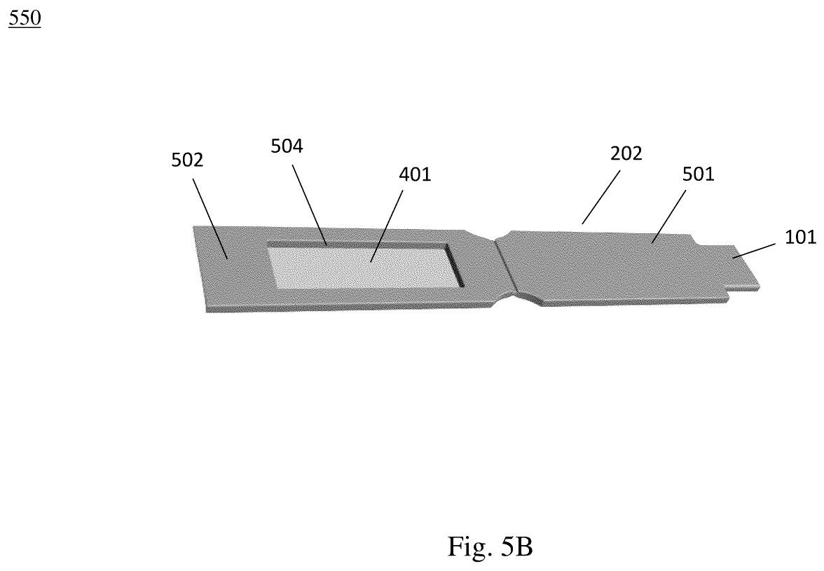

Referring now to FIGS. 5A-B, and in brief overview, views of another implementation of a collection strip 202 are shown that includes a first portion 501, a second portion 502, a breaking region 503, a window 504, a tab 101, and a sample membrane 401. The first portion 501 and the second portion 502 of the collection strip 202 are connected to each other by a breaking region 503 between them. A physical separation of the first portion 501 and second portion 502 can occur under certain conditions. The first portion 501 and the second portion 502 can be substantially rectangular. In some implementations, the first portion 501 and the second portion 502 can be different shapes such as rectangular, circular, triangular, or irregularly shaped. In some implementations, the first portion 501 and the second portion 502 can be different shapes from each other. The tab 101 can extend from an edge of the first portion 501 such that it is graspable by a user so that the user can extract the collection strip 202 from the multilayer collection tab by pulling on the tab 101. The window 504 can include a rectangular aperture within the second portion 502. The sample membrane 401 can be disposed on the top surface of the second portion 502, positioned over the window 504, which can allow visibility through the window 504 to the sample membrane 401. In some implementations, only the second portion 502 has a window 504 and a sample membrane 401 disposed on the top surface of the second portion 502, positioned over the window. In some implementations, only the first portion 501 has a window 504 and a sample membrane 401 disposed on the top surface of the first portion 501, positioned over the window.

In some implementations, the sample membrane 401 is a plurality of sample membranes. In some implementations, the window 504 is a plurality of windows. By way of example, the first portion 501 and the second portion 502 can each have a window 504 and a sample membrane 401 disposed on their top surface, positioned over their respective window. Each of the sample membranes 401 can be shaped and sized to extend past the perimeter of the window 504, with a spacing between adjacent sample membranes. In this way, the collection strip 202 can collect multiple samples on separate sample membranes, maximizing the collection surface area, and preventing direct contact between adjacent sample membranes.

At least one of the plurality of sample membranes 401 can be used to collect a whole blood sample, while at least one other of the plurality of sample membranes 401 can be used to collect a plasma sample. In some implementations, the sample membrane 401 can be formed of a multi-layer collection membrane that uses a plasma separating membrane which can allow plasma proteins and components to pass through while retaining blood cells.

Still referring to FIGS. 5A-B, and in greater detail, the breaking region 503 can include a region having a smaller thickness relative to the first portion 501 and the second portion 502. A thinner region between the first portion 501 and the second portion 502 can create a weaker zone within the collection strip 202 and induces a predetermined point of stress concentration, facilitating the separation of the portions 501,502 when force is applied. In some implementations, the transition from the thickness of the first portion 501 and second portion 502 to the reduced thickness of the breaking region 503 is gradual (e.g., a taper or gradient). In some implementations, the transition from the thickness of the first portion 501 and second portion 502 to the reduced thickness of the breaking region 503 is abrupt (e.g., a stepped change as illustrated in FIG. 5A). In some implementations, the breaking region 503 is a region having perforations which can include a series of small holes, slits, or indentations. The perforations can be arranged in a line pattern or other pattern between the first portion 501 and the second portion 502. In some implementations, the breaking region 503 is formed of a weak material (e.g., brittle material) that is prone to fracturing (e.g., breaking, tearing, ripping). A user can separate the first portion 501 and the second portion 502 by applying a bending and/or twisting force to the breaking region 503. A user can hold the collection strip 202 and position a distal end (e.g., the first portion 501 or the second portion 502) of the collection strip 202 into a receptacle and then apply a force to that region, allowing the distal end to separate and fall into the receptable while the proximal end (e.g., the first portion 501 or the second portion 502) of the collection strip 202 remains in the hand of the user. The proximal end can be placed into a slot of a receptable (e.g., primary container 803) for storing the collection strip 202.

In some implementations, the breaking region 503 is the same width as least one of the portions (e.g., the first portion 501, the second portion 502). In some implementations, the breaking region 503 is a region having a width smaller than the width of the two portions (e.g., the first portion 501, the second portion 502). In this way, the breaking region 503 can be more easily identified by the user. In some implementations, the collection strip 202 has an hourglass shape, with the narrowest width corresponding to the breaking region 503.

Still referring to FIGS. 5A-B, and in greater detail, the window 504 can be substantially rectangular. In some implementations, the window 504 can be different shapes such as rectangular, circular, triangular, or irregularly shaped. The window 504 can be configured to accommodate hole punching of the sample. In some implementations, one or more hydrophobic strips extend horizontally and/or vertically across at least one surface of the sample membrane 401, providing structural support to prevent the sample membrane 401 from breaking, tearing, ripping when stained with menstrual blood. In some implementations, the one or more hydrophobic strips are symmetrically positioned on both sides of the sample membrane 401. This symmetry can provide a visual aid to indicate areas where hole punching should be avoided.

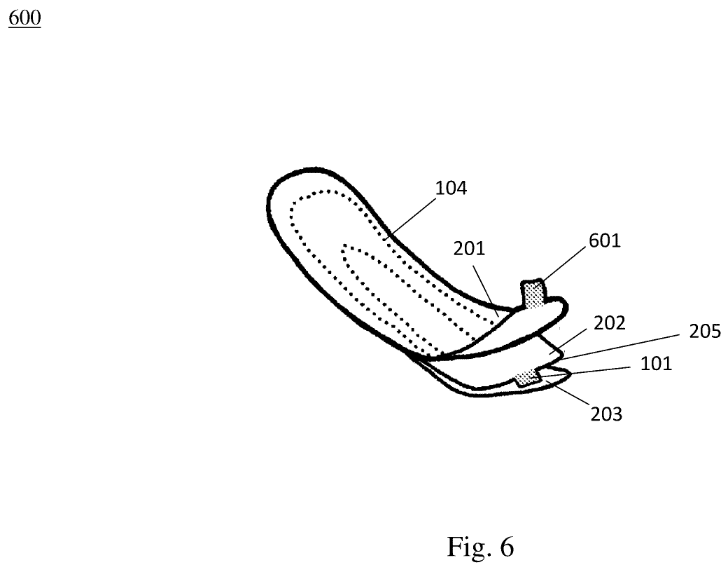

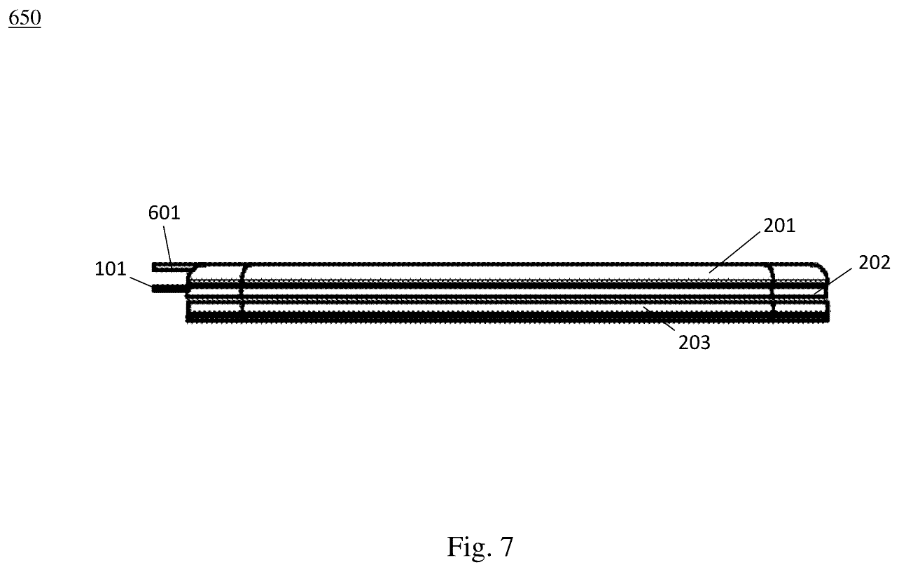

Referring now to FIG. 6, and in brief overview, and with additional reference to FIG. 7, a view of another implementation of a multilayer collection pad is shown that includes a top layer 201, a collection strip 202, and an adhesive layer 203. The top layer 201 can be configured to be in proximity to a flow of menstrual effluent of a user. The collection strip 202 can be disposed between the top layer 201 and the adhesive layer 203. The collection strip 202 can include a tab 101 extending from at least a portion of an edge 205 of the collection strip 202. The collection strip 202 can be formed of a material that undergoes staining when fluid contacts it. The tab 101 can extend past the perimeter of each of the layers. The top layer 201 can comprise a number of pores 104 with pore sizes selected to allow menstrual blood passage through the top layer while preventing the passage of eggs, mucus, blood clots, other non-blood related components, and other non-fluid effluent. The multilayer collection pad can further comprise a cover layer (e.g., cover layer 204) disposed below the adhesive layer 203. The collection strip 202 and the cover layer can be configured to be removable from the multilayer collection pad. The top layer 201 can further comprise a top layer tab 601. The top layer tab 601 can extend from an edge of the top layer 201 and can be substantially aligned with the tab 101 of the collection strip 202.

The tab 101 and the top layer tab 601 can be configured to be grabbable by a user such that the user can pull back the top layer 201 from the collection strip 202. The top layer 201 can be at least partially pulled back from the collection strip 202 to view the collection strip 202 and determine the degree of saturation of the collection strip 202. The top layer 201 can be completely pulled back from the collection strip 202 to extract the collection strip 202. In some implementations, the size and shape of the collection strip 202 corresponds to the size and shape of the adjacent layers (e.g., the top layer 201, the adhesive layer 203).

The multilayer collection pad can further comprise an adhesive disposed between the top layer 201, the collection strip 202, and the adhesive layer 203. The adhesive can comprise an adhesive configured to withstand repeated pulling and stretching with minimal loss of its adhesive properties. In this way, a user can repeatedly pull back the top layer 201 to visually check the saturation of the collection strip 202 throughout use until it is determined that the collection strip is sufficiently stained for biomarker testing. The collection strip 202 can be extracted from the adjacent layers by completely removing both the top layer 201 and the adhesive layer 203 from the collection strip 202. The adhesive can be any medical adhesive or skin-safe adhesive such as acrylic adhesives, silicone adhesives, hydrocolloid adhesives, polyurethane adhesives, and pressure-sensitive adhesives.

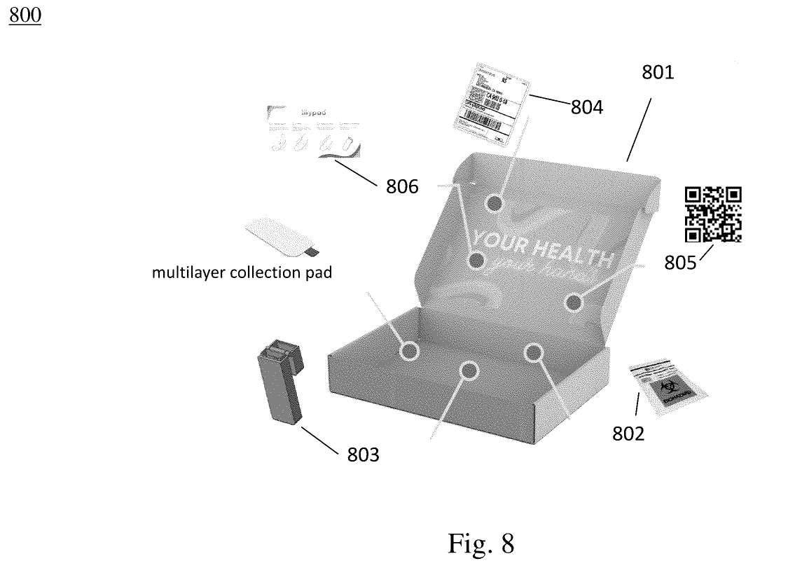

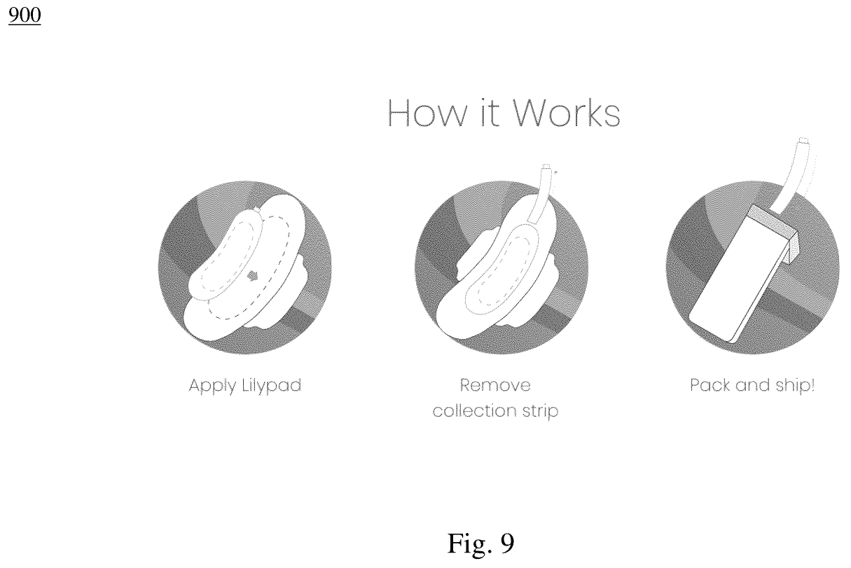

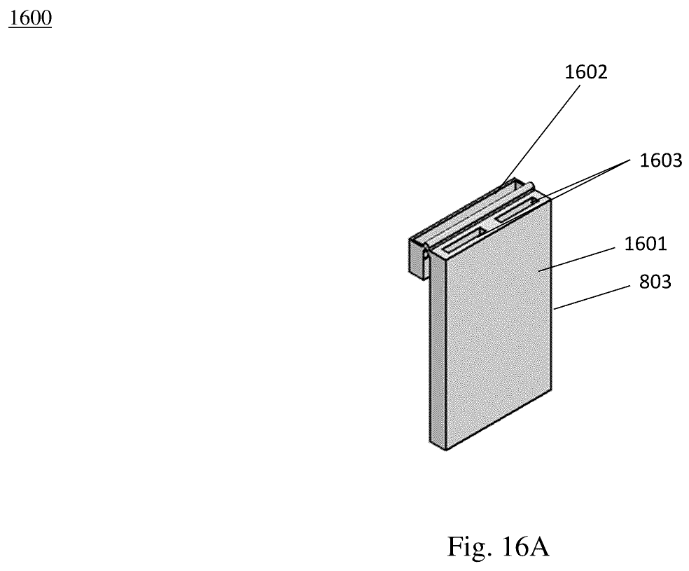

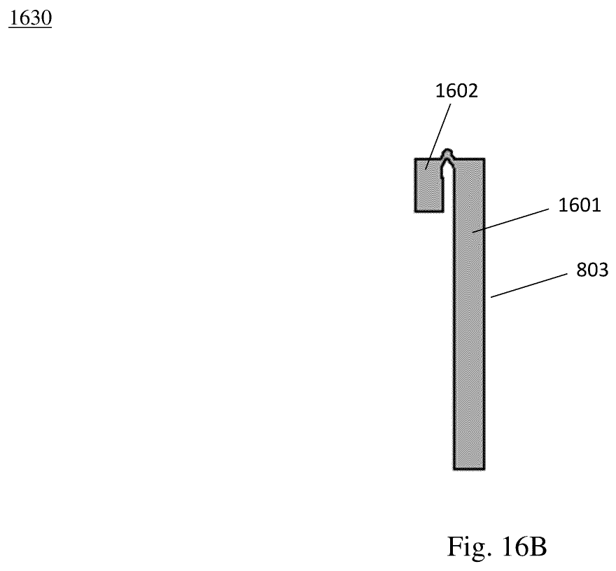

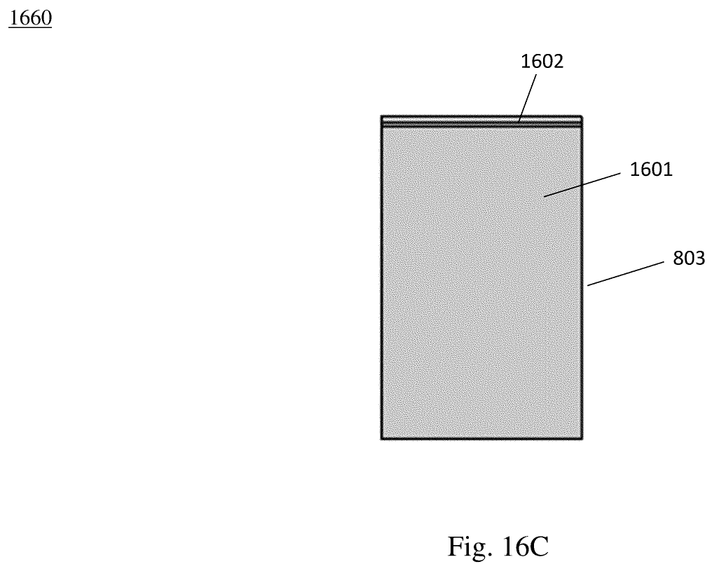

Referring now to FIG. 8, and with added reference to FIGS. 9 and 16A-C, in some implementations, the multilayer collection pad is comprised in a kit 801. The kit can further comprise a biosample bag 802, a primary container 803, a shipping label 804, a unique identification code 805, and user instructions 806. In some implementations, a plurality of collection pads can be comprised in the kit 801. The kit 801 can be shipped to the user or acquired at a store. In some implementations, the kit 801 can further comprise nitrile gloves and alcohol wipes. In some implementations, the biosample bag 802 contains a silica desiccant (e.g., silica gel packet). In some implementations, the primary container 803 contains silica desiccant.

Following the staining and removal of the collection strip 202 from the multilayer collection pad, the user can place the collection strip 202 into the primary container 803. The primary container can include a body 1601 and a top 1602 to seal a top end of the body 1601. The primary container 803 can be configured to dry the stained collection strip 202, maintain the collection strip 202 dry, and maintain a steady temperature and humidity level during shipment. In some implementations, the primary container 803 can comprise vents which enable fluid communication between the interior and exterior of the primary container. The primary container 803 can further include a plurality of slots 1603 to receive and secure at least one collection strip 202 during shipment. Each of the plurality of slots 1603 can include an opening and a channel that extends through a thickness of the body 1601. The opening can be different shapes such as a circle, rectangle, rectangle with rounded corners, triangle, or an irregular shape. The channel can be a circular channel, a rectangular channel, or an irregularly shaped channel. In some implementations, the primary container 803 is formed of a rigid material.

In some implementations, the biosample bag 802 can receive the primary container 803. After the user secures the at least one collection strip 202 in the primary container 803, the user can then seal the primary container 803 with the top 1602, as shown in the closed configuration of the primary container 802 shown on FIG. 16C. The primary container 803 can be placed into a biosample bag 802, which can be sealed as well. In other implementations, the user seals the primary container 803 and places it in a secondary container for shipping. In still other implementations, the user places the whole multilayer collection pad in a biosample bag and/or primary container 803 for shipping. The whole multilayer collection pad can be shipped for testing.

The kit 801 can further comprise a unique identification code 805. The unique identification code 805 can be disposed within the kit 801 such that it is only viewable by the user of the kit. The unique identification code 805 can include a serial code, a QR code, or a bar code. The user can scan the identification code 805 with a phone or can input a code provided by the unique identification code into an app-platform to review a status of the collection pad. A status of the collection pad can include a status of the shipping of the collection strip, a status of the laboratory processing of the collection strip, which can comprise, for example, the time until results are available, and the corresponding results. The collection strip 202 can further comprise a unique identification code so that a user or laboratory personnel handling the collection strip 202 can identify the collection strip 202.

The user instructions 806 can include readable and graphical instructions that instruct the user on application of the collection pad, usage of the collection pad, and shipping of the collection pad as shown in FIG. 8. In some implementations, the instructions for usage of the collection include a graphic illustrating an area of the collection to stain with menstrual blood of a user for processing and testing.

In some implementations, the kit 801 further comprises additional specimen collection tools including but not limited tools for collecting urine, saliva, and vaginal secretions. The kit 801 can contain a container configured for urine collection. The container can include a labeling to indicate the volume of urine required for biomarker testing. In some implementations, the kit 801 contains a collection card that provides an area for users to annotate the collection date and time of the sample. The user can saturate the collection card by dipping it into the urine sample and allow it to dry. Once dried, the collection card can be placed into a biohazard container for shipping. In some implementations, the collection card further comprises a unique identification code.

In some implementations, the kit 801 further comprises a vial for collecting saliva. The vial can include a labeling to indicate the volume of saliva required for biomarker testing. The vial can further include a capped opening in the shape of a funnel to facilitate easier collection. The user can collect saliva by passively drooling and/or spitting into the vial. In some implementations, the vial further comprises a unique identification code.

In some implementations, the kit 801 further comprises a specimen collection tool for collecting vaginal secretions which can include a cotton-tipped swab and a storage vial for storing the swab. The storage vial can contain a relatively small volume of ThinPrep solution or similar solutions to preserve the swab sample. The storage vial can contain a removable cap for sealing the sample.

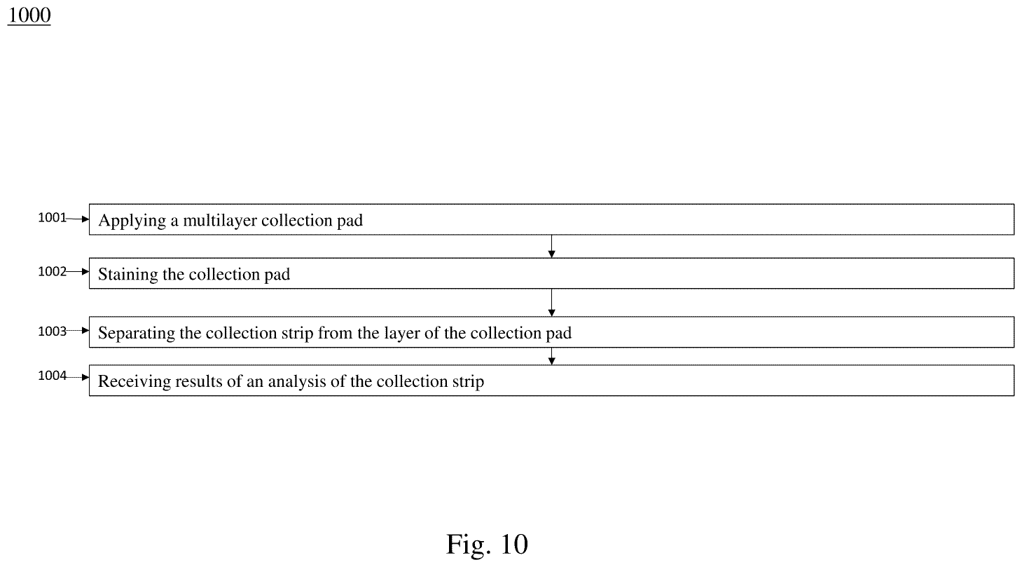

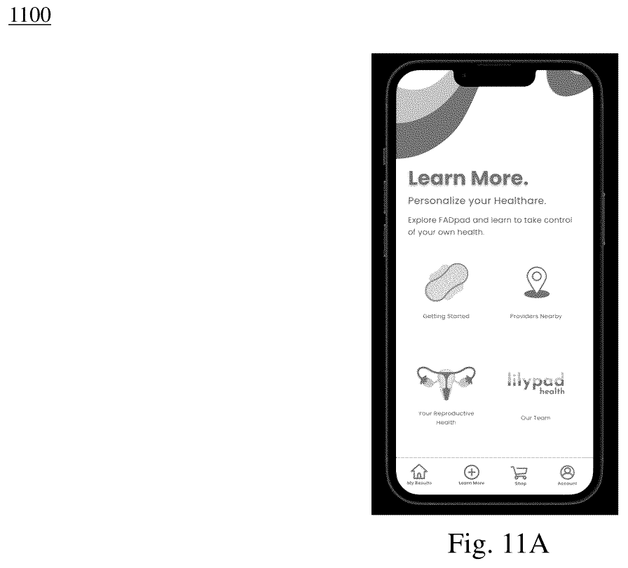

Referring now to FIG. 10, and in brief overview, a method of using an implementation of a multilayer collection pad is shown. The method includes: a step 1001, applying a multilayer collection pad, a step 1002, staining the collection pad, a step 1003, separating the collection strip from the layers of the collection pad, and a step 1004, receiving results of an analysis of the collection strip.

At step 1001, applying a multilayer collection pad, the multilayer collection pad can be applied to a fabric (e.g., undergarment, or on top of a menstrual pad). The step 1001, can further comprise removing the cover layer 204 of the multilayer collection pad to expose the underlying adhesive disposed on the base layer of the adhesive layer 203. The multilayer collection pad can be positioned and pressed on the fabric to adhere it to the fabric. The collection pad can be positioned so that it is in proximity to a flow of menstrual effluent of a user.

The method of using an implementation of a multilayer collection pad can further comprise a step of determining whether the collection pad is sufficiently stained for processing and testing. In some implementations, the top layer 201 of the multilayer collection pad can include an indicator 102. In those implementations, the user can visually check the top layer 201 to see whether menstrual blood fills the area outlined by the indicator 102. In other implementations, the user can visually compare the top layer 201 to the graphic of the user instructions 806 illustrating an area on the top layer 201 to stain with menstrual blood of a user for processing and testing. In still other implementations, the user can partially pull back the top layer 201 to view the staining on the collection strip 202. Determining whether the collection pad is sufficiently stained for processing and testing can be done during use of the multilayer collection pad (e.g., when the multilayer collection pad is still adhered to a fabric of the user).

At step 1003, separating the collection strip from the layers of the collection pad, can further comprise pulling on the tab 101 of the collection strip 202 and separating the collection strip 202 from the layers of the collection pad. The step 1003 can additionally or alternatively involve peeling or pulling off the layers adjacent to the collection strip 202 (e.g., the top layer 201, the adhesive layer 203) to separate the collection strip 202. In some implementations, the method can further comprise a step of separating the first portion 501 and the second portion 502 of the collection strip 202 by applying a force to the breaking region 503. In some implementations, the method can further comprise a step of processing and testing the collection strip 202 for biomarkers. This can include using laboratory testing methods, lateral flow assays, and/or rapid test assays.

In some implementations, the user receives results via mail. In other implementations, the user receives results electronically such as through e-mail, a website, or a mobile device application. The step 1004 can further comprise contacting a medical provider upon receiving results. In still other implementations, the user can receive results directly at their location through visible indicators displayed by a testing device (e.g., later flow assay device, rapid test assay device) used at their location after processing and testing the stained collection strip 202.

In some implementations, the method of using an implementation of a multilayer collection pad can further comprise shipping the collection strip 202. In other implementations, the method of using an implementation of a multilayer collection pad further comprises shipping the whole multilayer collection pad with the collection strip 202 still in place.

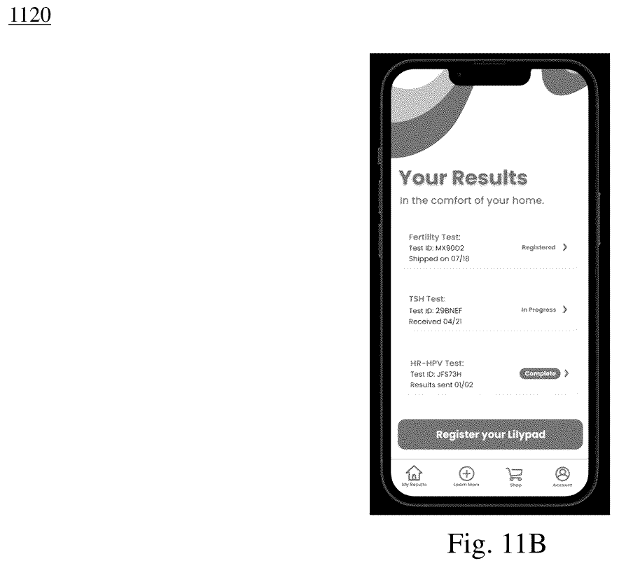

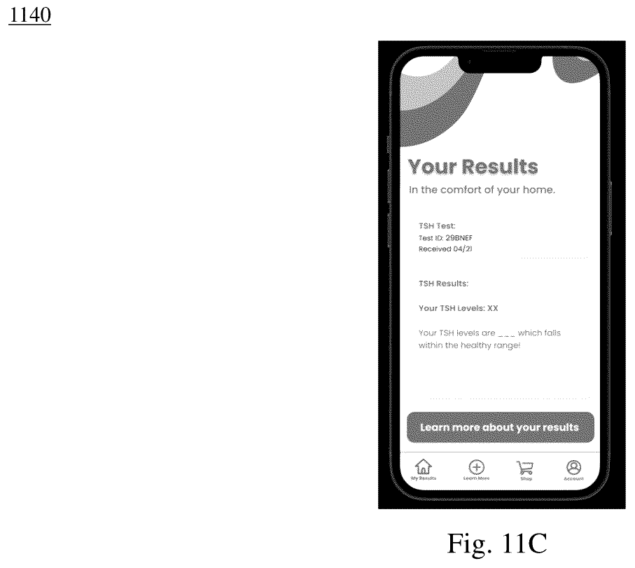

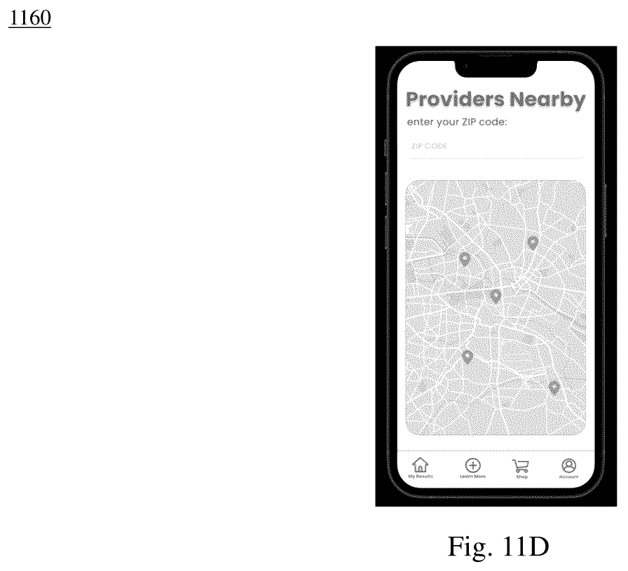

Referring now to FIGS. 11A-D, various views of a display screen of an implementation of an application (e.g., mobile device application) are shown. The application can be used to register a multilayer collection pad, receive results of an analyzed multilayer collection pad, contact medical providers, place orders, and/or access instructions/information. FIGS. 11B-C show examples of the status (e.g., “registered”, “in progress”, “complete”) and test results for various multilayer collection pad, along with their corresponding identification code. The application can provide a means for viewing results along with other information, such as information on how to understand the results, an interpretation of the results, and information for contacting medical providers for further care.

Referring now to FIGS. 12A-B, and in brief overview, and with additional reference to FIG. 12C, views of an implementation of a housing for lateral flow assay tests are shown. FIG. 12C shows a perspective view of the housing for lateral flow assay tests of FIGS. 12A-B with a collection strip 202 inserted through an opening 1206 of the housing. The housing can include a top cover 1201 and a base 1202. The top cover 1201 can include a well 1203 for receiving a buffer solution and a display window 1204. The base 1202 can include a recess 1205 which can define an opening 1206 between the top cover 1201 and the base 1202, and is configured to hold a collection strip 1202 within the recess 1204. The housing can contain a lateral flow assay for testing biomarkers, with the lateral flow assay disposed between the top cover 1201 and the base 1202, and oriented so that the result of the assay is visible through the display window 1204.

The well 1203 can include an aperture that extends through thickness of the top cover 1201 and is configured to receive a buffer solution. The buffer solution can flow through the well 1203 and contact the sample membrane 401 of a collection strip 202 disposed within the recess 1205. The buffer solution can be dispensed into the well 1203 to run the necessary antibodies/antigens through the embedded lateral flow assay. The display window 1204 can display one or more test indicators, which can allow for multiple tests to run on a single collection strip 202. Multiple tests can be performed using a multiplex lateral flow assay or a multianalyte later flow assay. These assays can detect and quantify multiple analytes or biomarkers from a single sample. They can use different regions on the same assay to provide simultaneous results for multiple indicators.

In some implementations, the collection strip 202 can be tested on a lateral flow assay device containing a lateral flow assay. The stained sample membrane 401 can be placed into a first solution. The first solution can contain a sample diluent solution with a buffer solution for proteins and antibodies. In some implementations, the stained sample membrane 401 is completely removed from the collection strip 202 and then deposited in the first solution. In other implementations, the complete collection strip 202 (with the stained sample membrane 401) is deposited in the first solution. In still other implementations, the first portion 501 and the second portion 502 of the collection strip 202 are separated from each other and the portion containing the stained sample membrane 401 is deposited in the first solution. The stained sample membrane 401 can then be submerged within the first solution and agitated or stirred in the solution for a specified duration of time. The first solution can then be dispensed into a well of the lateral flow assay device. A second solution can then be dispensed into a well of the lateral flow assay device. The second solution can contain a running buffer which can buffer the sample's pH, determine the flow speed, reduce non-specific binding, and nullify interfering components present within the sample. The first and second solution can be contained within a bottle configured to dispense solution onto a lateral flow assay device.

In some implementations, the collection strip 202 can be tested with rapid tests assays. After removing the collection strip 202 from the multilayer collection pad, the stained sample membrane 401 can be placed into a first solution. The first solution can contain a sample diluent solution with a buffer solution for proteins and antibodies. In some implementations, the stained sample membrane 401 is completely removed from the collection strip 202 and then deposited in the first solution. In other implementations, the complete collection strip 202 (with the stained sample membrane 401) is deposited in the first solution. In still other implementations, the first portion 501 and the second portion 502 of the collection strip 202 are separated from each other and the portion containing the stained sample membrane 401 is deposited in the first solution. The stained sample membrane 401 can then be submerged within the first solution and agitated or stirred for a specified duration. After a specified duration, a second solution can be agitated and dispensed onto the rapid test device. The second solution can contain a color developer. In following, a third solution can be dispensed onto the rapid test device after a specified duration. The third solution can contain a clarifying solution configured to make the test indicators on the rapid test easier to read.

The first, second, and third solution can be contained within a bottle configured to dispense solution onto a rapid test device containing a rapid test assay. The first solution can contain a buffer solution with cell lysis reagents. The stained sample membrane 401 can be added to the first solution to lyse blood cells collected by the sample membrane 401. The second solution can contain a solution that reacts with captured antibodies/antigens to produce a colored mark at the control indicator. If target antibodies/antigens are present within the sample, a colored mark is also produced at the test indicator.

The rapid test device can include a housing and a test membrane contained within the housing. The test membrane can be made of a nitrocellulose filtration membrane on top of an absorbent material. The test membrane can be treated with proteins that react with target antibodies/antigens to produce a visual signal.

Test assays such as rapid test assays and lateral flow assay tests can be performed by the user, for example, at home, or by laboratory personnel. Results of the rapid test assay and later flow assay test can include a negative, positive, or invalid result which is indicated by a visible indicator that appears after running the assay. A visible control indicator alone indicates a negative result; a visible control and test indicator indicate a positive result when target biomarkers are present in the sample (e.g., antibodies/antigens). Any other variation indicates an invalid test.

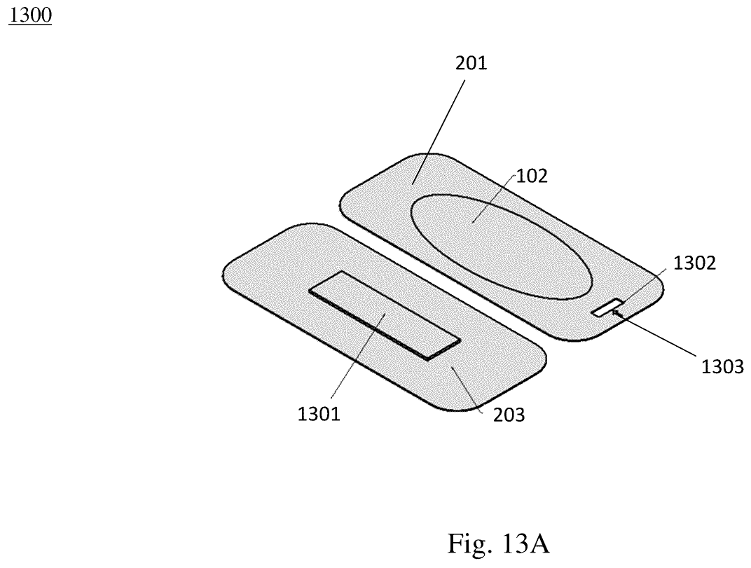

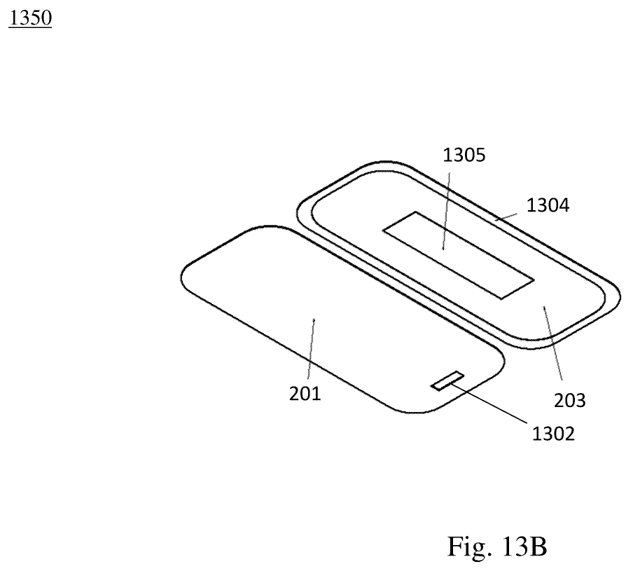

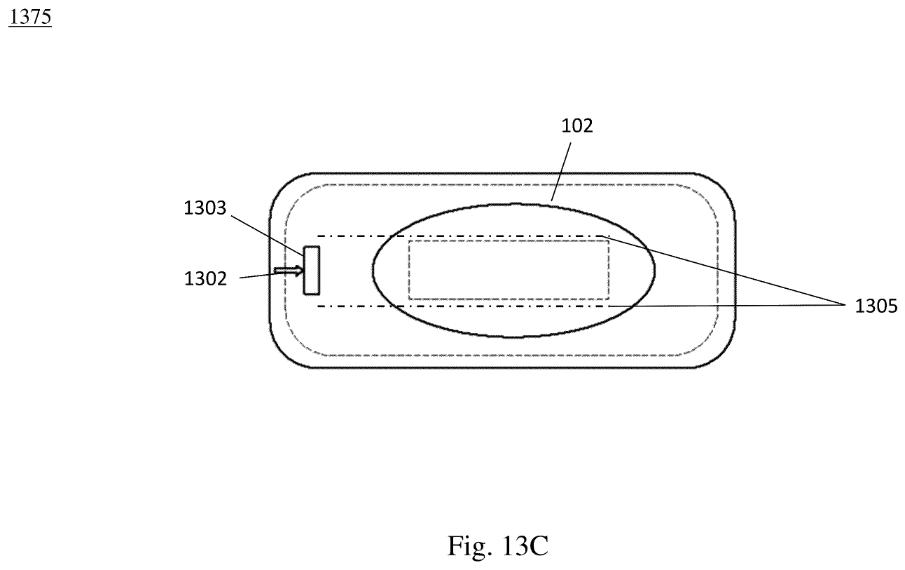

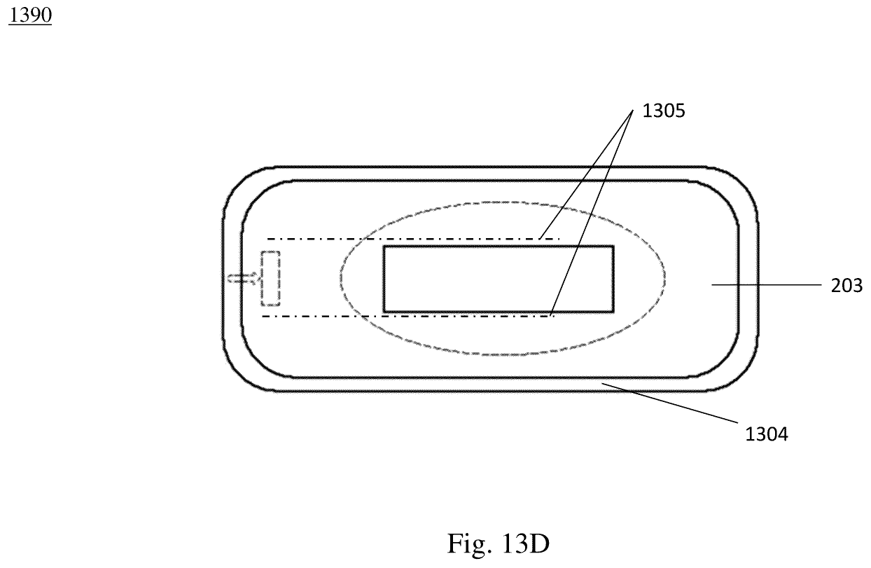

Referring now to FIGS. 13A-D, and in brief overview, various views of another implementation of a multilayer layer collection pad are shown including a top layer 201, an adhesive layer 203, and a sponge membrane 1301. The top layer 201 can be configured to be in proximity to a flow of menstrual effluent of a user and fluid permeable to menstrual blood. The sponge membrane 1301 can be configured to absorb menstrual blood. The sponge membrane 1301 can be disposed between the top layer 201 and the adhesive layer 203. In some implementations, the sponge membrane 1301 can be at least partially exposed through the top layer 201. In these implementations, the top layer 201 can comprise an opening positioned over the sponge membrane 1301, such that the opening is shaped and sized to expose at least a portion of the sponge membrane 1301. The adhesive layer 203 can be configured to be in proximity to a fabric (e.g., menstrual pad beneath the multilayer collection pad, undergarment) of a user. The adhesive layer 203 can include a fluid impermeable region 1305 (as shown in FIG. 13B) positioned underneath the sponge membrane 1301. This can help facilitate blood sample collection by ensuring that when a swab is pressed against the sponge membrane 1301, it efficiently absorbs the blood without leakage or interference.

Still referring to FIGS. 13A-D, and in greater detail, the top layer 201, the adhesive layer 203, and the sponge membrane 1301 can form a multilayer stack in which the perimeters of at least the top layer 201 and the adhesive layer 203 are substantially aligned. In some implementations, the multilayer layer collection pad can further comprise a cover layer such that the adhesive layer 203 is disposed between the top layer 201 and the cover layer (e.g., cover layer 204), and the sponge membrane disposed between the top layer 201 and the adhesive layer 203. The cover layer can be configured to be removable from the multilayer collection pad.

The top layer 201, the adhesive layer 203, and the cover layer 204 can form a multilayer stack with the collection strip 202 such that the perimeters of at least the layers 201, 203, 204 are substantially aligned. The top layer 201 and the adhesive layer 203 can be coupled to each other. In some implementations, the top layer 201 and the adhesive layer 203 can be coupled to each other (e.g., via heat sealing) along a portion of their perimeter such that an unsealed portion (e.g., unsealed portion 105) provides an opening through which a swab can be inserted through. In other implementations, the top layer 201 and the adhesive layer 203 can be coupled to each other (e.g., via heat sealing) along their entire perimeter. In these implementations, the top layer 201 can comprise an insertion slit 1302 that extends through the thickness of the top layer 201. The insertion slit 1302 can be different shapes such as a circle, rectangle, rectangle with rounded corners, triangle, or an irregular shape. The top layer 201 can further comprise an indicator 1303 to indicate to the user the location of the insertion slit 1302. The user can insert the head of a swab through the insertion slit 1302 to contact the sponge membrane 1301. The insertion slit 1302 can include a border. The border can be formed of a fluid impermeable and/or hydrophobic material which can prevent the insertion slit 1302 from sticking to the sponge membrane 1301 or from closing, thereby ensuring that users can easily access the sponge membrane 1301 with a swab.

The multilayer collection pad can further comprise a slit path 1305. The slit path 1305 can include 2 boundary lines that extend through the layers above and below the sponge membrane 1301 (e.g., top layer 201, adhesive layer 203) which can help direct the swab to the sponge membrane 1301. The two boundary lines can be substantially parallel to each other, and the distance between them can correspond to the width of the sponge membrane 1301, and can have an additional tolerance. These two boundary lines can extend from the insertion slit 1302 to a perpendicular edge of the sponge membrane 1301. It should be understood that depending on the shape and dimensions of the sponge membrane 1301, the slit path 1305 may vary to correspond to the geometry of the sponge membrane 1301.

By extending through all the layers of the multilayer collection pad, the slit path 1305 can define a pocket configured to hold the sponge membrane 1301 in place, ensuring proper alignment and preventing shifting of the sponge membrane 1301. In some implementations, the pocket defined by the slit path 1305 comprises a fluid impermeable material and/or hydrophobic material to prevent sticking of the top layer 201 to the sponge membrane 1301.

After swabbing the sponge membrane 1301, the swab can be placed in a primary container (e.g., primary container 803) and/or a biohazard bag and shipped to a lab for testing. The primary container 803 can be configured to dry the swab, maintain the swab dry, and maintain a steady temperature and humidity level during shipment. The primary container 803 can include a plurality of slots 1603 to receive and secure at least one swab during shipment. Each of the plurality of slots 1603 can include an opening and a channel that extends through a thickness of the body 1601. The opening can be different shapes such as a circle, rectangle, rectangle with rounded corners, triangle, or an irregular shape. The channel can be a circular channel, a rectangular channel, or an irregularly shaped channel. In some implementations, the plurality of slots 1603 contain a buffer solution.

The sponge membrane 1301 can be formed of medical sponge material used to collect blood samples. The sponge membrane 1301 can be configured to absorb menstrual blood and release menstrual blood when the head of a swab contacts the sponge membrane 1301. The sponge membrane 1301 can be made of one or more materials (e.g., a combination thereof), including but not limited to polyurethane foam, sea sponge, polypropylene, polyvinyl alcohol, silicone, cellulose.

The top layer 201 can be a fluid permeable top layer that makes contact with the user's skin and receives the flow of menstrual effluent of the user. The top layer 201 can filter menstrual effluent (e.g., menstrual blood, vaginal secretions) to ensure that menstrual blood stains the sponge membrane 1301 (e.g., the menstrual blood saturates the sponge membrane 1301 and the surrounding layers). Menstrual blood can include whole blood, plasma, DNA, RNA, proteins, antigens, antibodies, cells, and other disease biomarkers.

The top layer 201 can be formed of a material that allows fluid communication between the menstrual blood of the user and the sponge membrane 1301. The top layer 201 can comprise a material that is porous and defines a number of pores through which menstrual blood can flow through so that blood can contact and stain the sponge membrane 1301. Excess menstrual blood that flows through the top layer 201 (i.e., blood that does not stain the sponge membrane 1301) can flow through to the adhesive layer 203.

The top layer 201 can comprise a number of pores (e.g., pores 104) with pore sizes selected to allow menstrual blood passage through the top layer while preventing the passage of eggs, mucus, blood clots, other non-blood related components, and other non-fluid effluent.

The top layer 201 can further include an indicator 102. The indicator 102 can include a marked outlined perimeter that is sized and shaped to demonstrate to the user an area to fill with menstrual blood. The size of the area enclosed by the outlined perimeter can correspond to the degree of saturation of the top layer 201 pad necessary to saturate the sponge membrane 1301 with sufficient menstrual blood for processing and biomarker testing.

In some implementations, the multilayer collection pad can further comprise an interior layer. The interior layer can be disposed between the top layer 201 and the adhesive layer 203. The interior layer can include an acquisition distribution layer (ADL) configured to rapidly absorb menstrual blood. This can quickly transfer the blood through to the adhesive layer 203, allowing it to flow through the permeable regions of the adhesive layer 203 and to a fabric disposed beneath the multilayer collection pad. This can help prevent the top surface of the multilayer layer collection pad from remaining a wet surface, ensuring a more comfortable experience for the user.

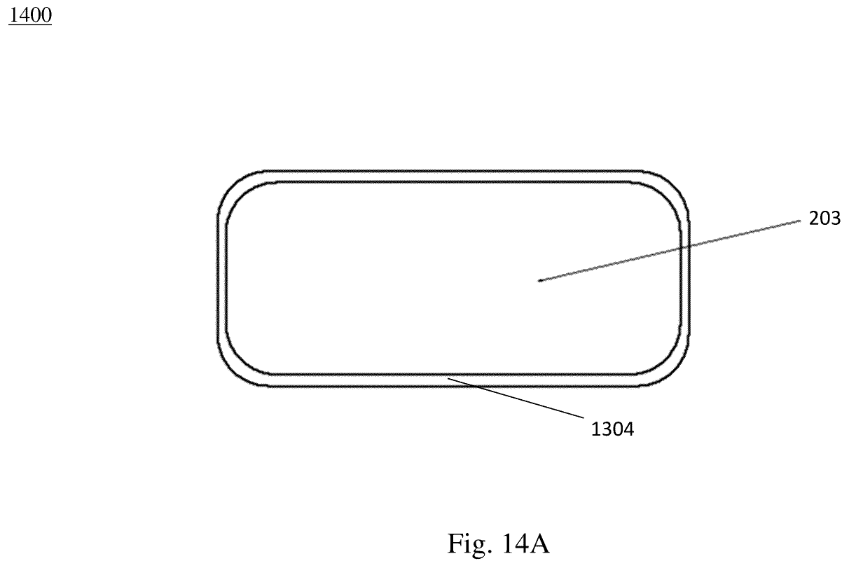

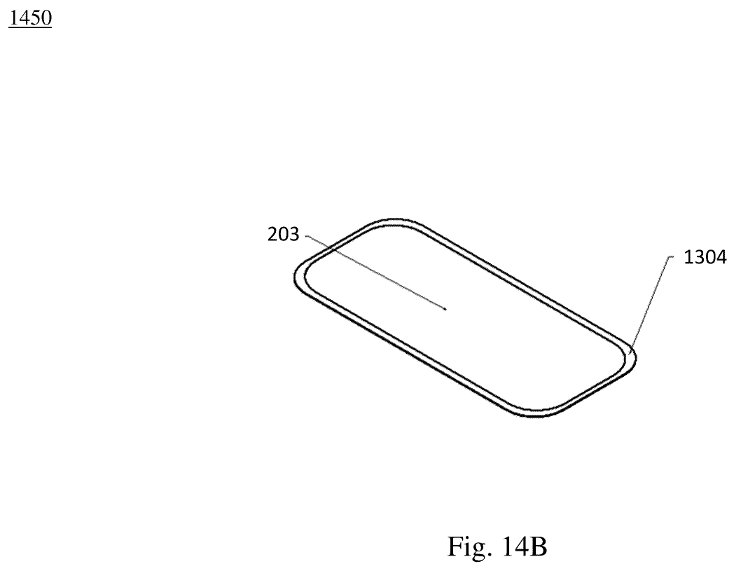

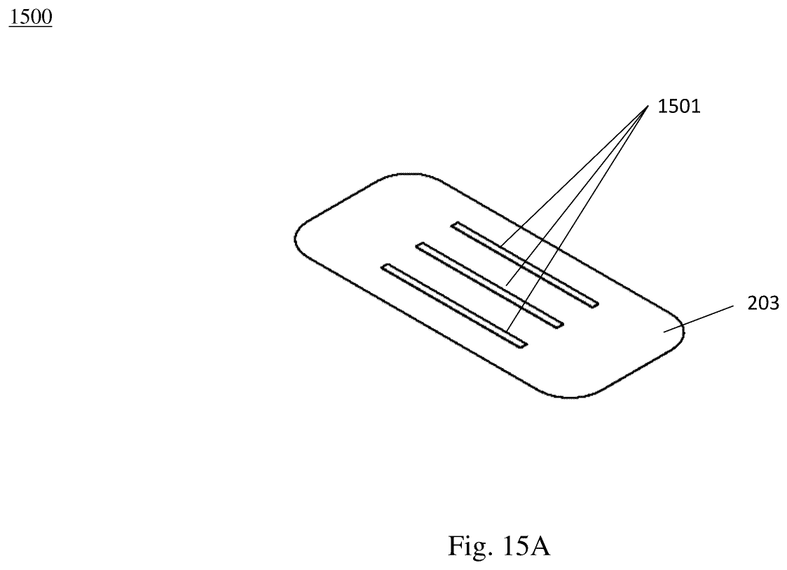

Still referring to FIGS. 13A-D, and with added reference to FIGS. 14A-B, the adhesive layer 203 can include a base layer, an adhesive ring 1304, and adhesive. The adhesive can be disposed on the adhesive ring 1304 and can be disposed so that it is proximal to a fabric of the user (e.g., undergarment, menstrual pad beneath the multilayer collection pad) when the user applies the multilayer collection pad. The adhesive layer 203 can adhere the multilayer collection pad to the menstrual pad or undergarment of a user. In some implementations the adhesive layer 203 at least partially absorbs the menstrual blood of the user. The adhesive ring 1304 can be formed of a fluid impermeable material.

The swab can include a fluid impermeable shaft and an absorbent head to collect a blood sample. The absorbent head can be made of one or more materials (e.g., a combination thereof), including but not limited to cotton, rayon, polyester, or foam. The swab can be a flocked swab which may be spray coated with short Nylon fibers. The fluid impermeable shaft can be made of one or more materials (e.g., a combination thereof), including but not limited polystyrene, plastic, wood, or paper.

A method of manufacturing a multilayer collection pad is provided. The multilayer collection pad can be manufactured using conventional processes commonly employed in the manufacturing of multilayer menstrual pads to securely bond layers of the pad to each other. One or more processes can be used to bond the layers (e.g., the top layer 201, the adhesive layer 203, additional layers) of the multilayer collection pad to each other. These techniques and processes can include, but are not limited to, thermal pressing, ultrasonic bonding, adhesive application, and rotary press processing. Thermal pressing can include using heat and pressure to bond layers to each other. In some implementations, a pneumatic thermal press (e.g., Sonitek Heat Staker) can be used for sealing both rigid and flexible materials of various shapes. The pneumatic thermal press can operate with control over temperature, dwell time, and pressure, ensuring bonding conditions for different material types and configurations. Ultrasonic bonding can include using high-frequency sound waves to create localized heat and pressure to bond the layers to each other. Adhesive application can include applying adhesive to attach the layers to each other. Adhesives can include but are not limited to hot melt adhesives, pressure-sensitive adhesives, and water-based adhesives. Rotary press processing can include a rotary press system. A rotary press system can use rotating cylinders or roller to perform various tasks, such as printing, stamping, or shaping materials. The rotary press system can be used to assemble the multilayer collection pad, combining the layers and adhering them together. The rotary press system can be configured to assemble the individual layers of the multilayer collection pad, cut the shape of the layers, and bond the layers to each other along their perimeter. The top layer 201 and the adhesive layer 203 can be heat pressed together along a portion of their perimeter such that an unsealed portion 105 provides an opening through which the collection strip 202 can be inserted through. The tab 101 of the collection strip 202 can extend through the unsealed portion 105 when in place.

One or more processes (e.g., thermal pressing, ultrasonic bonding, adhesive application, and rotary press processing) can be used to bond the layers above and below the collection strip 202 and along the collection strip boundary 106. In this way, the collection strip boundary 106 can extend through the layers (e.g., the top layer 201, the adhesive layer 203) of the multilayer collection pad to define a pocket configured to hold the collection strip 202 in place.

One or more processes (e.g., thermal pressing, ultrasonic bonding, adhesive application, and rotary press processing) can be used to bond the layers above and below the sponge membrane 1301 and along the slit path 1305. In this way, the slit path 1305 can extend through the layers (e.g., the top layer 201, the adhesive layer 203) of the multilayer collection pad to define a pocket configured to hold the sponge membrane 1301 in place and guide the insertion of a path.

The collection strip 202 can be manufactured using one or more processes, including but not limited to extrusion, roll slitting, die-cutting, laser cutting, water jet cutting. Extrusion can include forcing molten plastic through a die to create continuous strips of uniform thickness. The continuous strips can then be cool and cut to the desired size and shape. Roll slitting can include unwinding a large roll of sheeting material and passing it through rotating knives or laser cutters that cut the sheet into strip of the desired size and shape. Die cutting can include using a custom-designed die to stamp out strips from a sheet of material. Laser cutting can include using a high-powered laser to precisely cut through a sheet of material.

The sample membrane 401 can then be adhered to a surface of the collection strip 202, overlaying a window 504, in such a way so that the sample membrane 401 remains stationary on the collection strip 202 and does not shift when a pressure or force is applied. The sample membrane 401 can be applied to the collection strip 202 using one or more processes, including but not limited to adhesive application, ultrasonic bonding, and thermal pressing the sample membrane 401 to the collection strip 202 with a plastic, film, or similar material along the border of the sample membrane 401.

The collection strip 202 can be inserted through the pocket defined by the collection strip boundary 106 using an inline pick-and-place system. An inline pick-and-place system is a type of automated machinery used in manufacturing and assembly processes. The system can operate by picking items (e.g., the collection strip 202, the multilayer collection pad) from a supply area placing them into specific positions or onto other components in a linear or sequential manner. The system can use a robotic arm, gripper, or vacuum system to pick items.

In some implementations, the rotary press system can be used with an inline pick-and-place system and inline platen press packaging system. An inline platen press packaging system is a specialized type of machinery that can package items at high speeds and continuously using a platen mechanism. An inline platen press packaging system can be used individually package the multilayer collection pad. Individually packaging each multilayer collection pad can protect the pad from damage during shipping to a user's location.

The indicator 102 can be added to the top layer 201 using a dye or by embossing the shape and lettering of the indicator 102 into the material of the top layer 201.

The disclosed system may be embodied in other specific forms without departing from the spirit or essential characteristics thereof. The foregoing implementation are therefore to be considered in all respects illustrative, rather than limiting of the invention. Having thus described several illustrative implementation, it is to be appreciated that various alterations, modifications, and improvements will readily occur to those skilled in the art. Such alterations, modifications, and improvements are intended to form a part of this disclosure, and are intended to be within the spirit and scope of this disclosure. While some examples presented herein involve specific combinations of functions or structural elements, it should be understood that those functions and elements may be combined in other ways according to the present disclosure to accomplish the same or different objectives. In particular, acts, elements, and features discussed in connection with one implementation are not intended to be excluded from similar or other roles in other implementations. Additionally, elements and components described herein may be further divided into additional components or joined together to form fewer components for performing the same functions. Accordingly, the foregoing description and attached drawings arc by way of example only, and are not intended to be limiting.

1. A multilayer collection pad comprising:
a fluid permeable top layer configured to be in proximity to a flow of menstrual effluent of a user, the menstrual effluent of a user comprising menstrual blood;
a cover layer; and
an adhesive layer disposed between the fluid permeable top layer and the cover layer, such that a perimeter of each of the layers is substantially aligned;
a collection strip disposed between the fluid permeable top layer and the adhesive layer, the collection strip including a tab extending from an edge of the collection strip past the perimeter of each of the layers.

2. The multilayer collection pad of claim 1, further comprising a non-organic layer disposed between the collection strip and the adhesive layer.

3. The multilayer collection pad of claim 1, the collection strip comprising organic material.

4. The multilayer collection pad of claim 1, the fluid permeable top layer further comprising a tab extending past the perimeter of each of the plurality of layers.

5. The multilayer collection pad of claim 1, wherein the adhesive layer comprises a base layer and an adhesive disposed on at least a portion of the base layer.

6. The multilayer collection pad of claim 5, the adhesive comprising hydrophobic material.

7. The multilayer collection pad of claim 5, the base layer comprising hydrophilic material.

8. The multilayer collection pad of claim 1, the fluid permeable top layer comprising a number of pores, each of the number of pores having a diameter in the range of 0.1 to 25 micrometer.

9. The multilayer collection pad of claim 1, the adhesive layer comprising a number of pores, each of the number of pores having a diameter in the range of 0.1 to 25 micrometer, such that the adhesive layer is permeable to menstrual blood of the user.

10. The multilayer collection pad of claim 1, the cover layer covering the adhesive.

11. The multilayer collection pad of claim 1, the fluid permeable top layer further comprising an indicator, the indicator sized and shaped to demonstrate to the user of the multilayer collection pad an area to fill with menstrual blood.

12. A kit comprising:
a multilayer collection pad comprising:
a fluid permeable top layer configured to be in proximity to a flow of menstrual effluent of a user, the menstrual effluent of a user comprising menstrual blood;
a cover layer; and
an adhesive layer disposed between the fluid permeable top layer and the cover layer, such that a perimeter of each of the layers is substantially aligned;
a collection strip disposed between the fluid permeable top layer and the adhesive layer, the collection strip including a tab extending from an edge of the collection strip past the perimeter of each of the layers; and
a primary container for receiving the collection strip.

13. The kit of claim 12, the primary container further comprising silica desiccant.

14. The kit of claim 12, the primary container further comprising a plurality of slots to secure at least one collection strip.

15. The kit of claim 12, further comprising a unique identification code for reviewing a status of the collection pad.

16. The kit of claim 15, the collection strip further comprising the unique identification code.

17. The kit of claim 15, the unique identification code comprising one of a serial code or a QR code.

18. The kit of claim 15, the unique identification code comprising of a QR code.

19. The kit of claim 12, comprising readable and graphical instructions including:
application of the collection pad;
usage of the collection pad, the instructions for usage of the collection pad including a graphic illustrating an area of the collection pad to stain with menstrual blood of a user for laboratory testing; and
shipping of the collection strip.

20. A method of using a multilayer collection pad comprising:
applying a multilayer collection pad comprising:
a fluid permeable top layer configured to be in proximity to a flow of menstrual effluent of a user, the menstrual effluent of a user comprising menstrual blood,
a cover layer; and
an adhesive layer disposed between the fluid permeable top layer and the cover layer, such that a perimeter of each of the layers is substantially aligned;
a collection strip disposed between the fluid permeable top layer and the adhesive layer, the collection strip including a tab extending from an edge of the collection strip past the perimeter of each of the layers;
staining the collection pad;
separating the collection strip from the layers of the collection pad; and
receiving results of an analysis of the collection strip.

21. The method of claim 20, further comprising:
determining whether the collection pad is sufficiently stained for testing.
